(19) United States
(12) Patent Application Publication
(10) Pub. No.: US 2026/0168003 A1
(43) Pub. Date: Jun. 18, 2026

(54) METHOD AND SYSTEM FOR PRODUCING A MODIFIED PROTEIN

(71) Applicant: YEDA RESEARCH AND DEVELOPMENT CO. LTD., Rehovot (IL)

(72) Inventors: Sarel-Jacob FLEISHMAN, Rehovot (IL); Lucas KRAUSS, Rehovot (IL)

(21) Appl. No.: 19/422,655

(22) Filed: Dec. 17, 2025

Related U.S. Application Data
(60) Provisional application No. 63/734,982, filed on Dec. 17, 2024.

(51) Int. Cl. C12P 21/02; C07K 14/445; G16B 35/10; G16B 35/20

(52) U.S. Cl. CPC C12P 21/02; C07K 14/445; G16B 35/10; G16B 35/20

(57) ABSTRACT

The present invention relates to biological sciences, specifically to the design and stabilization of proteins for use in diagnostics and vaccine development. The present invention represents a system and method for producing a modified protein that provides an improvement of the technological field of protein design and stabilization by providing tools that enable the efficient design and production of proteins with enhanced stability characteristics compared to the parental protein, while retaining immunogenicity to naturally acquired human monoclonal antibodies. In particular, the suggested invention is efficiently applicable to proteins with large and topologically complex structures that have limited natural diversity of homologs and circumvent the requirement for a deep multiple-sequence alignment.

### CROSS-REFERENCE TO RELATED APPLICATIONS

This application claims the benefit of priority to U.S. Provisional Application No. 63/734,982, titled “METHOD AND SYSTEM FOR PRODUCING A MODIFIED PROTEIN”, filed Dec. 17, 2024, which is hereby incorporated by reference in its entirety.

### FIELD OF THE INVENTION

The present invention relates to biological sciences, specifically to the design and stabilization of proteins for use in diagnostics and vaccine development.

### BACKGROUND OF THE INVENTION

Designing stable and highly expressible proteins for use in diagnostics and therapeutics is a significant challenge in the field of biological sciences. Known methods for protein stabilization have shown success in introducing multiple stabilizing mutations. However, these methods face limitations when applied to proteins having a limited natural diversity of homologous sequences. The need to maintain the antigenic profile of proteins while enhancing their stability and expression levels further complicates the design process.

This problem is particularly evident in the context of malaria diagnostics and vaccine development.

Malaria remains a significant global health challenge, particularly in regions outside of Africa where Plasmodium vivax is the predominant species causing the disease. P. vivax infections often relapse due to the reactivation of dormant liver stage parasites known as hypnozoites. These hypnozoites serve as a reservoir for transmission but are undetectable by current commercial diagnostic tests. Antibodies against the P. vivax Reticulocyte Binding Protein 2b (PvRBP2b ) have emerged as reliable serological biomarkers for recent infections, providing indirect indicators for the risk of relapse.

Despite the potential of PvRBP2b as a diagnostic marker, the application of PvRBP2b has been limited by low expression yields and stability in recombinant microbial systems. Existing computational and experimental approaches to enhance protein stability and expressibility often introduce stabilizing mutations at solvent-exposed positions. For PvRBP2b, a significant portion of the solvent-accessible surface is targeted by naturally acquired human antibodies, necessitating a design strategy that maintains the antigenic profile while introducing mutations to improve stability.

Known natural homolog-based protein stability-design methods are not commonly applied to design diagnostic antigens in which surface mutations are strongly limited. A further complication is that these methods usually rely on a multiple-sequence alignment of homologs to restrict atomistic design choices to amino acid identities that are commonly observed in the natural diversity. This aspect of the known methodology is critical to its ability to design dozens of simultaneous mutations in large and complex proteins without leading to aggregation, misfolding, and loss of function. In particular, the limited natural diversity of PvRBP2b homologs further complicates the application of these methods, as the conventional PSSM (Position-Specific Scoring Matrix) derived from sequence homologs may not be sufficiently accurate.

Known AI-based sequence design tools were successfully applied to de novo designs, the majority of which were based on small α-helical structures However, the applications of these tools to natural proteins (which are typically larger and topologically more complex than de novo designed ones) showed lower success rates than known natural homolog-based methods and required significant experimental screening, which would be impractical in the case of a complex protein such as PvRBP2b.

### SUMMARY OF THE INVENTION

Accordingly, there is a need for a system and method for producing a modified protein that would provide an improvement of the technological field of protein design and stabilization by providing tools that enable the efficient design and production of proteins with enhanced stability characteristics compared to the parental protein, while retaining immunogenicity to naturally acquired human monoclonal antibodies. In particular, such tools should be efficiently applicable to proteins with large and topologically complex structures that have limited natural diversity of homologs and circumvent the requirement for a deep multiple-sequence alignment. Such tools would potentially help overcoming barriers to the implementation of economical and resilient diagnostics and vaccine immunogens for many infectious diseases.

To address the aforementioned needs, the following is suggested.

In the general aspect, the invention may be directed to a method of producing a modified protein comprising at least one modified polypeptide chain having amino acid substitutions relative to an original polypeptide chain in a parental protein. The suggested method may include: generating a protein expression vector suitable for use in a protein expression system and expressing the modified protein in said protein expression system using said protein expression vector, thereby producing the modified protein. Generating the protein expression vector suitable for use in the protein expression system may be preformed by: (i) determining a plurality of backbone features characterizing a backbone of the parental protein or a segment thereof; (ii) inferring a pretrained encoder-decoder-based message passing neural network (MPNN) on the determined plurality of backbone features to predict a probability for an amino acid to be located at a specific position of the backbone; (iii) based on the predicted probability, defining a position-specific scoring matrix (PSSM) characterizing, per each position in the original polypeptide chain, amino acid alternatives; (iv) combinatorically generating a plurality of designed sequences, each of said designed sequences corresponds to a modified polypeptide chain and comprises one or more amino acid substitutions each being one of said amino acid alternatives, and threading each of said designed sequences on a template structure of said original polypeptide chain, to thereby generate a plurality of designed structures; (v) sorting said plurality of designed structures according to a minimized energy scoring, said minimized energy scoring is determined by subjecting each of said designed structures to an energy minimization; and (vi) selecting at least one of said plurality of designed structures, corresponding to said modified polypeptide chain, based on said minimized energy scoring, thereby obtaining an amino acid sequence of said modified polypeptide chain for use as sequence data input for providing said protein expression vector.

In another general aspect, the present invention may be directed to a system for producing a modified protein comprising at least one modified polypeptide chain having amino acid substitutions relative to an original polypeptide chain in a parental protein. The system may include: at least one non-transitory memory device, wherein modules of instruction code are stored, and at least one processor associated with said at least one memory device, and configured to execute the modules of instruction code, whereupon execution of said modules of instruction code, the at least one processor may be configured to: generate a protein expression vector suitable for use in a protein expression system; and express the modified protein in said protein expression system using said protein expression vector, thereby producing the modified protein. Said at least one processor may be configured to generate the protein expression vector suitable for use in the protein expression system by: (i) determining a plurality of backbone features characterizing a backbone of the parental protein or a segment thereof; (ii) inferring a pretrained encoder-decoder-based message passing neural network (MPNN) on the determined plurality of backbone features to predict a probability for an amino acid to be located at a specific position of the backbone; (iii) based on the predicted probability, defining a position-specific scoring matrix (PSSM) characterizing, per each position in the original polypeptide chain, amino acid alternatives; (iv) combinatorically generating a plurality of designed sequences, each of said designed sequences corresponds to a modified polypeptide chain and comprises one or more amino acid substitutions each being one of said amino acid alternatives, and threading each of said designed sequences on a template structure of said original polypeptide chain, to thereby generate a plurality of designed structures; (v) sorting said plurality of designed structures according to a minimized energy scoring, said minimized energy scoring is determined by subjecting each of said designed structures to an energy minimization; and (vi) selecting at least one of said plurality of designed structures, corresponding to said modified polypeptide chain, based on said minimized energy scoring, thereby obtaining an amino acid sequence of said modified polypeptide chain for use as sequence data input for providing said protein expression vector.

In some embodiments, said substitutions improve the stability of the modified protein relative to the parental protein, as determined by at least one of: a thermal denaturation temperature of the modified protein being equal or higher than a thermal denaturation temperature of the parental protein; a solubility of the modified protein being equal or higher than a solubility of the parental protein; a degree of misfolding of the modified protein being equal or lower than a degree of misfolding of the parental protein; a half-life of the modified protein being equal or longer than a half-life of the parental protein; a specific activity of the modified protein being equal or higher than a specific activity of the parental protein; and a recombinant expression level of the modified protein being equal or higher than a recombinant expression level of the parental protein.

In some embodiments, said determining a plurality of backbone features characterizing a backbone of the parental protein may include: determining the coordinates of one or more backbone atoms of a protein, wherein the backbone atoms include at least one of: a nitrogen (N) atom, a carbon (C) atom, an oxygen (O) atom, an alpha carbon (Cα) atom, and a virtual beta carbon (Cβ); based on the determined coordinates, constructing a graph representation of the backbone, said graph representation having nodes representing amino acids of the parental protein, and edges defined by the backbone features, said backbone features being formed based on at least one of: interatomic distances related to adjacent nodes; characteristics of relative atomic frame orientations and rotations between adjacent nodes; backbone dihedral angles in the adjacent nodes.

In some embodiments, said inferring the MPNN may include: preprocessing the constructed graph representation to make the nodes thereof dissociated from the amino acids of the parental protein; using a message-passing algorithm to encode the pre-processed graph representation, thereby obtaining an encoded graph representation comprising latent information about a structure of the backbone, said message-passing algorithm having messages constructed by applying a pretrained multilayer perception (MLP) model on each pair of the adjacent nodes and edges therebetween; applying a decoding algorithm on the encoded graph representation, to predict, per each node and each amino acid, a probability of a respective node to be associated with a respective amino acid, thereby predicting the probability for the amino acid to be located at the specific position of the backbone.

In some embodiments said defining the PSSM may include: logarithmically normalizing the predicted probabilities; averaging the normalized probabilities over a plurality of independent initializations, to account for stochastic variations; and defining the PSSM based on the averaged normalized probabilities.

In some embodiments, a selected modified polypeptide chain may correspond to designed structure having a minimal value for said minimized energy scoring.

In some embodiments, said energy minimization may be a global energy minimization.

In some embodiments, said plurality of designed sequences may be combinatorically generated under an acceptance threshold based on said stability scoring.

In some embodiments, said at least one modified polypeptide chain may include at least six amino acid substitutions relative to said original polypeptide chain.

In some embodiments, said energy minimization may include at least one operation selected from the group consisting of bond length optimization, bond angle optimization, backbone dihedral angles optimization, amino acid side-chain packing optimization, rigid-body optimization, and sidechain dihedral angle minimizations of the modified polypeptide chain.

In some embodiments, said at least one processor may be configured to determine a plurality of backbone features characterizing a backbone of the parental protein by: determining the coordinates of one or more backbone atoms of a protein, wherein the backbone atoms include at least one of: a nitrogen (N) atom, a carbon (C) atom, an oxygen (O) atom, an alpha carbon (Cα) atom, and a virtual beta carbon (Cβ); and, based on the determined coordinates, constructing a graph representation of the backbone, said graph representation having nodes representing amino acids of the parental protein, and edges defined by the backbone features, said backbone features being formed based on at least one of: interatomic distances related to adjacent nodes; characteristics of relative atomic frame orientations and rotations between adjacent nodes; backbone dihedral angles in the adjacent nodes.

In some embodiments, said at least one processor may be configured to infer the MPNN by: preprocessing the constructed graph representation to make the nodes thereof dissociated from the amino acids of the parental protein; using a message-passing algorithm to encode the pre-processed graph representation, thereby obtaining an encoded graph representation comprising latent information about a structure of the backbone, said message-passing algorithm having messages constructed by applying a pretrained multilayer perception (MLP) model on each pair of the adjacent nodes and edges therebetween; and applying a decoding algorithm on the encoded graph representation, to predict, per each node and each amino acid, a probability of a respective node to be associated with a respective amino acid, thereby predicting the probability for the amino acid to be located at the specific position of the backbone.

In some embodiments, said at least one processor may be configured to define the PSSM by: logarithmically normalizing the predicted probabilities; averaging the normalized probabilities over a plurality of independent initializations, to account for stochastic variations; and defining the PSSM based on the averaged normalized probabilities.

In some embodiments, said at least one processor may be further configured to perform the energy minimization by at least one operation selected from the group consisting: of bond length optimization, bond angle optimization, backbone dihedral angles optimization, amino acid side-chain packing optimization, rigid-body optimization, and sidechain dihedral angle minimization of the modified polypeptide chain.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 depicts a block diagram of a computing device that may be included in a system for producing a modified protein, and configured to execute instruction code for performing steps of the methods described herein, according to some embodiments of the present invention;

FIG. 2 depicts a block diagram of the system for producing a modified protein, according to some embodiments of the present invention;

FIG. 3 is a flow diagram depicting a method for producing a modified protein, according to some embodiments of the present invention;

FIG. 4 represents a design process of stabilized PvRBP2b₁₆₉₋₄₇₀ variants, according to some embodiments;

FIG. 5A illustrates a Coomassie-stained SDS-PAGE gel showing Ni-NTA affinity-purified samples of the original polypeptide chain (PvRBP2b169-470) and nine stabilized designs under reducing conditions, as obtained according to some embodiments of the present invention;

FIG. 5B shows a bar graph comparing final protein yields (mg/L of bacterial culture) for the original polypeptide chain and stabilized designs after sequential purification steps (Ni-NTA affinity, ion exchange, and size exclusion chromatography), as obtained according to some embodiments of the present invention;

FIG. 5C presents a table summarizing dynamic light scattering (DLS) measurements for the original polypeptide chain and three stabilized designs, including hydrodynamic radius (Rh) and unfolding onset temperature (Tonset) values, highlighting enhanced thermal stability in the designs, as obtained according to some embodiments of the present invention;

FIG. 6A is a schematic structural overlay illustrating crystal structures of WHT2483 (purple) and WHT2484 (green) are overlayed with parental PvRBP2b₁₆₉₋₄₇₀ with RMSD values provided, as obtained according to some embodiments of the present invention;

FIG. 6B is a schematic representation of mutated residues shown as spheres mapped onto ribbon representations of the parental PvRBP2b₁₆₉₋₄₇₀ structure, as obtained according to some embodiments of the present invention;

FIG. 6C is a schematic surface representation depicting binding footprints of human antibodies against parental PvRBP2b₁₆₉₋₄₇₀, as obtained according to some embodiments of the present invention;

FIG. 6D is a schematic surface representation showing total antibody bound surface (deep blue) for the eight human monoclonal antibodies, shown on parental PvRBP2b₁₆₉₋₄₇₀ surface (white), as obtained according to some embodiments of the present invention;

FIG. 7A is a graphical representation in the form of boxplots representing antibody responses towards PvRBP2b proteins measured in relative antibody units (RAU), as obtained according to some embodiments of the present invention;

FIG. 7B is a scatter plot representation showing the correlation between antibody responses to the parental PvRBP2b169-470 protein and stabilized designs, as obtained according to some embodiments of the present invention;

FIG. 7C is a graphical representation in the form of receiver operating characteristic (ROC) curves for eight-antigen sero-diagnostic combination comparing parental PvRBP2b and designs, and the associated area under the curve (AUC), as obtained according to some embodiments of the present invention;

FIG. 8A is a graphical representation comprising ion exchange chromatography (IEX) chromatograms and corresponding reduced SDS-PAGE gels of parental PvRBP2b₁₆₉₋₄₇₀ and designs, as obtained according to some embodiments of the present invention;

FIG. 8B is a graphical representation comprising size exclusion chromatography (SEC) chromatograms and corresponding reduced SDS-PAGE gels of parental PvRBP2b₁₆₉₋₄₇₀ and designs, as obtained according to some embodiments of the present invention;

FIG. 8C is a gel electrophoresis representation showing non-reduced (NR) and reduced (R) SDS-PAGE gel of recombinant parental PvRBP2b₁₆₉₋₄₇₀ and designs after affinity, IEX and SEC purification steps, as obtained according to some embodiments of the present invention;

FIG. 8D is a table showing inflection temperatures (Ti) of parental PvRBP2b₁₆₉₋₄₇₀ and designs using label free thermal shift analysis (Tycho NT.6, Nanotemper), as obtained according to some embodiments of the present invention;

FIG. 9 represents electrostatics surfaces of PvRBP2b₁₆₉₋₄₇₀ and stabilized designs, as obtained according to some embodiments of the present invention;

FIG. 10 represents representative BLI binding curves with PvRBP2b₁₆₉₋₄₇₀ designs and human monoclonal antibodies, as obtained according to some embodiments of the present invention;

FIG. 11 provides Table 1 representing mutated residues between parental PvRBP2b₁₆₉₋₄₇₀ and designs, as obtained according to some embodiments of the present invention;

FIG. 12 provides Table S1 representing mutated residues between parental PvRBP2b₁₆₉₋₄₇₀ and designs, as obtained according to some embodiments of the present invention;

FIG. 13 provides Table S2 representing data collection and refinement statistics for WHT2483 and WHT2484, as obtained according to some embodiments of the present invention;

FIG. 14 provides Table S3 representing P. vivax proteins utilized in Luminex assays and the amounts coupled, as obtained according to some embodiments of the present invention;

FIG. 15 provides Table S4 representing top performing combination of antigens in random forest classification algorithm when using parental PvRBP2b compared to stabilized designs.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

One skilled in the art will realize the invention may be embodied in other specific forms without departing from the spirit or essential characteristics thereof. The foregoing embodiments are therefore to be considered in all respects illustrative rather than limiting of the invention described herein. Scope of the invention is thus indicated by the appended claims, rather than by the foregoing description, and all changes that come within the meaning and range of equivalency of the claims are therefore intended to be embraced therein.

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention. Some features or elements described with respect to one embodiment may be combined with features or elements described with respect to other embodiments. For the sake of clarity, discussion of same or similar features or elements may not be repeated.

Although embodiments of the invention are not limited in this regard, discussions utilizing terms such as, for example, “processing,” “computing,” “calculating,” “determining,” “establishing”, “analyzing”, “checking”, “choosing”, “selecting”, “omitting”, “training”, “applying”, “forming” or the like, may refer to operation(s) and/or process(es) of a computer, a computing platform, a computing system, or other electronic computing device, that manipulates and/or transforms data represented as physical (e.g., electronic) quantities within the computer's registers and/or memories into other data similarly represented as physical quantities within the computer's registers and/or memories or other information non-transitory storage medium that may store instructions to perform operations and/or processes.

Although embodiments of the invention are not limited in this regard, the terms “plurality” and “a plurality” as used herein may include, for example, “multiple” or “two or more”. The terms “plurality” or “a plurality” may be used throughout the specification to describe two or more components, devices, elements, units, parameters, or the like. The term “set” when used herein may include one or more items.

In some sections, the present description refers to natural homolog-based protein stability-design methods and aspects thereof. It shall be understood that, depending on the embodiments of the present invention, any known natural homolog-based protein stability-design methods, or specific aspects thereof, may be applied herein, and it will be clear, for the person skilled in the art, how to apply these methods or aspects thereof in order to implement the present invention. As a non-exclusive example, known natural homolog-based protein stability-design method may be or may include aspects of PROSS atomistic design method.

In some sections, the present description refers to AI-based sequence design tools and aspects thereof. It shall be understood that, depending on the embodiments of the present invention, any known AI-based sequence design tools, or specific aspects thereof, may be applied herein, and it will be clear, for the person skilled in the art, how to apply these tools or aspects thereof in order to implement the present invention. As a non-exclusive example, known AI-based sequence design tool may be or may include aspects of ProteinMPNN sequence design tool.

Unless explicitly stated, the method embodiments described herein are not constrained to a particular order or sequence. Additionally, some of the described method embodiments or elements thereof can occur or be performed simultaneously, at the same point in time, concurrently, or iteratively and repeatedly.

In embodiments of the present invention, some steps of the claimed method may be performed using machine-learning (ML)-based models or may include actions performed on ML-based models, e.g., transferring ML-based models over a computer network. ML-based models may be configured or “trained” for a specific task, e.g., classification or regression.

In some embodiments, ML-based models may be artificial neural networks (ANN).

A neural network (NN) or an artificial neural network (ANN), e.g., a neural network implementing a machine learning (ML) or artificial intelligence (AI) function, may refer to an information processing paradigm that may include nodes, referred to as neurons, organized into layers, with links between the neurons. The links may transfer signals between neurons and may be associated with weights. A NN may be configured or trained for a specific task, e.g., pattern recognition or classification. Training a NN for the specific task may involve adjusting these weights based on examples. Each neuron of an intermediate or last layer may receive an input signal, e.g., a weighted sum of output signals from other neurons, and may process the input signal using a linear or nonlinear function (e.g., an activation function). The results of the input and intermediate layers may be transferred to other neurons and the results of the output layer may be provided as the output of the NN. Typically, the neurons and links within a NN are represented by mathematical constructs, such as activation functions and matrices of data elements and weights. A processor, e.g., CPUs or graphics processing units (GPUs), or a dedicated hardware device may perform the relevant calculations.

It should be obvious for the one ordinarily skilled in the art that various ML-based models can be implemented without departing from the essence of the present invention. It should also be understood, that in some embodiments ML-based model may be a single ML-based model or a set (ensemble) of ML-based models realizing as a whole the same function as a single one. Hence, in view of the scope of the present invention, the abovementioned variants should be considered equivalent.

According to the concept of the present invention, the suggested is a systematic approach to producing a modified protein with enhanced stability and expression levels. By integrating a pretrained encoder-decoder-based message passing neural network (MPNN) with the atomistic design workflow that is similar to the one applied in the natural homolog-based protein stability-design methods, the method allows for the generation of a position-specific scoring matrix (PSSM) that accurately predicts amino acid substitutions, regardless of the limited natural diversity of homologs of the parental protein, such as PvRBP2b. Accordingly, this approach circumvents the need for a deep multiple-sequence alignment.

The method involves determining backbone features of the parental protein, using these features to construct a graph representation, and applying the MPNN to predict the probability of amino acids at specific positions. This results in a PSSM that guides the generation of designed sequences with amino acid substitutions. These sequences are then threaded onto a template structure and subjected to energy minimization to identify the most stable configurations.

This innovative combination of AI-based and atomistic design calculations ensures that the modified proteins retain their antigenic properties while achieving higher expression yields and thermal stability. The practical application of this method is demonstrated by the production of stabilized PvRBP2b variants that maintain binding to naturally acquired human monoclonal antibodies, making them suitable for use in diagnostics and vaccine development. This approach addresses the limitations of traditional methods and provides a reliable solution for enhancing the biophysical properties of proteins critical for infectious disease diagnostics and therapeutics.

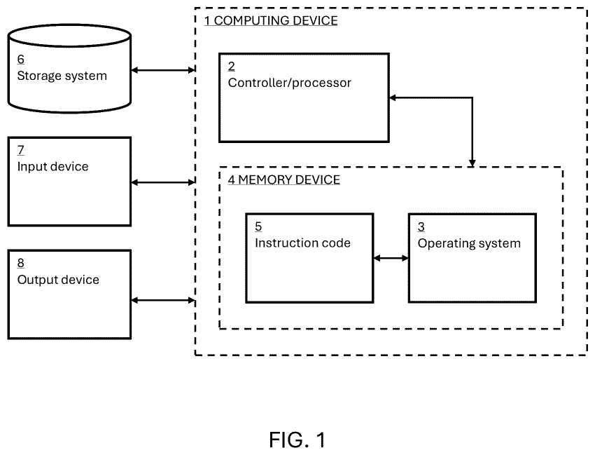

Reference is now made to FIG. 1, which is a block diagram depicting a computing device, which may be included within an embodiment of a system for producing a modified protein, according to some embodiments.

Computing device 1 may include a processor or controller 2 that may be, for example, a central processing unit (CPU) processor, a chip or any suitable computing or computational device, an operating system 3, a memory device 4, instruction code 5, a storage system 6, input devices 7 and output devices 8. Processor 2 (or one or more controllers or processors, possibly across multiple units or devices) may be configured to carry out methods described herein, and/or to execute or act as the various modules, units, etc. More than one computing device 1 may be included in, and one or more computing devices 1 may act as the components of, a system according to embodiments of the invention.

Operating system 3 may be or may include any code segment (e.g., one similar to instruction code 5 described herein) designed and/or configured to perform tasks involving coordination, scheduling, arbitration, supervising, controlling or otherwise managing operation of computing device 1, for example, scheduling execution of software programs or tasks or enabling software programs or other modules or units to communicate. Operating system 3 may be a commercial operating system. It should be noted that an operating system 3 may be an optional component, e.g., in some embodiments, a system may include a computing device that does not require or include an operating system 3.

Memory device 4 may be or may include, for example, a Random-Access Memory (RAM), a read only memory (ROM), a Dynamic RAM (DRAM), a Synchronous DRAM (SD-RAM), a double data rate (DDR) memory chip, a Flash memory, a volatile memory, a non-volatile memory, a cache memory, a buffer, a short-term memory unit, a long-term memory unit, or other suitable memory units or storage units. Memory device 4 may be or may include a plurality of possibly different memory units. Memory device 4 may be a computer or processor non-transitory readable medium, or a computer non-transitory storage medium, e.g., a RAM. In one embodiment, a non-transitory storage medium such as memory device 4, a hard disk drive, another storage device, etc. may store instructions or code which when executed by a processor may cause the processor to carry out methods as described herein.

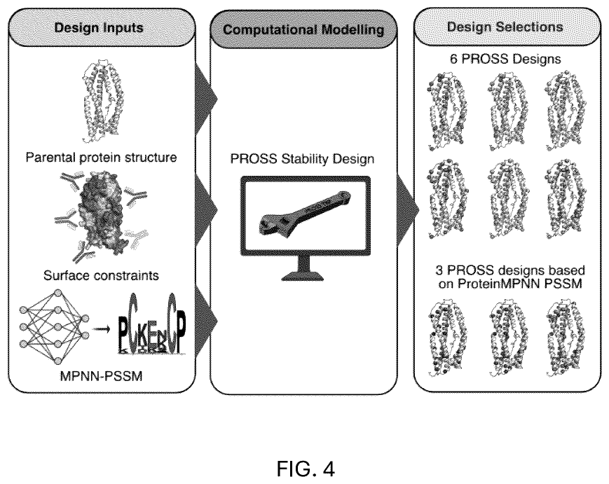

Instruction code 5 may be any executable code, e.g., an application, a program, a process, task, or script. Instruction code 5 may be executed by processor or controller 2 possibly under control of operating system 3. For example, instruction code 5 may be a standalone application or a software module that may be configured to perform: generating a protein expression vector suitable for use in a protein expression system by: (i) determining a plurality of backbone features characterizing a backbone of the parental protein or a segment thereof; (ii) inferring a pretrained encoder-decoder-based message passing neural network (MPNN) on the determined plurality of backbone features to predict a probability for an amino acid to be located at a specific position of the backbone; (iii) based on the predicted probability, defining a position-specific scoring matrix (PSSM) characterizing, per each position in the original polypeptide chain, amino acid alternatives; (iv) combinatorically generating a plurality of designed sequences, each of said designed sequences corresponds to a modified polypeptide chain and comprises one or more amino acid substitutions each being one of said amino acid alternatives, and threading each of said designed sequences on a template structure of said original polypeptide chain, to thereby generate a plurality of designed structures; (v) sorting said plurality of designed structures according to a minimized energy scoring, said minimized energy scoring is determined by subjecting each of said designed structures to an energy minimization; and (vi) selecting at least one of said plurality of designed structures, corresponding to said modified polypeptide chain, based on said minimized energy scoring, thereby obtaining an amino acid sequence of said modified polypeptide chain for use as sequence data input for providing said protein expression vector; and expressing the modified protein in said protein expression system using said protein expression vector, thereby producing the modified protein. Although, for the sake of clarity, a single item of instruction code 5 is shown in FIG. 1, a system according to some embodiments of the invention may include a plurality of executable code segments or modules similar to instruction code 5 (e.g., backbone feature extractor 110, encoder-decoder-based message passing neural network 120, PSSM generator 130, sequence design module 140, structure modeling module 150, energy minimization module 160, selection and output module 170, expression vector construction module 180 and protein expression system 190, as shown, e.g., in FIG. 4) that may be loaded into memory device 4 and cause processor 2 to carry out methods described herein.

Storage system 6 may be or may include, for example, a flash memory as known in the art, a memory that is internal to, or embedded in, a micro controller or chip as known in the art, a hard disk drive, a CD-Recordable (CD-R) drive, a Blu-ray disk (BD), a universal serial bus (USB) device or other suitable removable and/or fixed storage unit. Various types of input and output data may be stored in storage system 6 and may be loaded from storage system 6 into memory device 4 where it may be processed by processor or controller 2. In some embodiments, some of the components shown in FIG. 1 may be omitted. For example, memory device 4 may be a non-volatile memory having the storage capacity of storage system 6. Accordingly, although shown as a separate component, storage system 6 may be embedded or included in memory device 4.

Input devices 7 may be or may include any suitable input devices, components, or systems, e.g., a detachable keyboard or keypad, a mouse, a touchscreen and the like. Output devices 8 may include one or more (possibly detachable) displays or monitors, speakers and/or any other suitable output devices. Any applicable input/output (I/O) devices may be connected to computing device 1 as shown by blocks 7 and 8. For example, a wired or wireless network interface card (NIC), a universal serial bus (USB) device or external hard drive may be included in input devices 7 and/or output devices 8. It will be recognized that any suitable number of input devices 7 and output device 8 may be operatively connected to computing device 1 as shown by blocks 7 and 8.

A system according to some embodiments of the invention may include components such as, but not limited to, a plurality of central processing units (CPU) or any other suitable multi-purpose or specific processors or controllers (e.g., similar to element 2), a plurality of input units, a plurality of output units, a plurality of memory units, and a plurality of storage units.

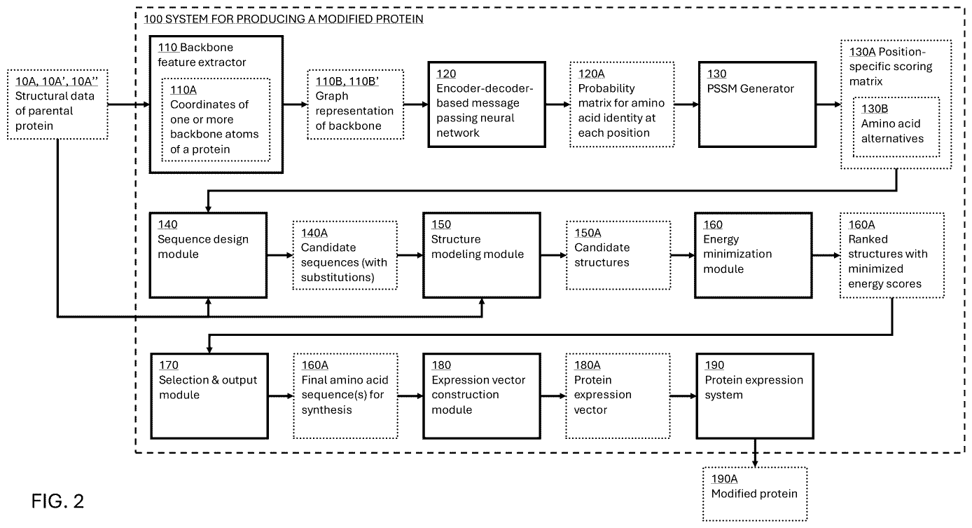

Reference is now made to FIG. 2, which is a block diagram of system 100 for producing a modified protein, according to some embodiments of the present invention.

According to some embodiments of the invention, system 100 may be implemented as a software module, a hardware module, or a combination thereof. For example, system 100 may be or may include computing devices such as element 1 of FIG. 1, or similar thereto. Components of system 100 may be adapted to execute one or more modules of instruction code (e.g., element 5 of FIG. 1) to request, receive, analyze, calculate and produce various data.

As further described in detail herein, system 100 may be adapted to execute one or more modules of instruction code (e.g., element 5 of FIG. 1) in order to perform steps of the claimed method.

As shown in FIG. 2, arrows may represent flow of one or more data elements to and from system 100 and/or among modules or elements of system 100. Some arrows may have been omitted in FIG. 2 for the purpose of clarity.

In some embodiments, system 100 may include backbone feature extractor 110. Backbone feature extractor 110 may be configured to receive structural data of parental protein 10A.

As used herein, structural data 10A represents coordinate level information describing the backbone of a parental protein (or a segment thereof).

In some embodiments, structural data 10A comprises atom coordinates (Cartesian 3D, typically in Ångströms) for backbone atoms including N, Cα, C, and O; and in certain embodiments, virtual Cβ coordinates derived from N—Cα-C geometry. In some embodiments, structural data 10A further comprises residue indexing (sequence position, chain identifier, insertion codes, alternate locations, occupancy, B factors). In some embodiments, data 10A further comprises secondary structure annotations (optional; e.g., from DSSP or in file HELIX/SHEET records).

In some embodiments, structural data 10A may be provided in at least one of PDB, mmCIF, or a serialized tensor format, and may be derived from experimentally determined structures (e.g., X ray crystallography, cryo EM, NMR) or from computational predictions (e.g., AlphaFold or equivalent). Structural data 10A may represent the full parental protein or a designable fragment (e.g., residues 169-470 of PvRBP2b), and, in certain implementations, may include multiple conformers (e.g., alternate chain models or biological assemblies).

In some embodiments, backbone feature extractor 110 may be configured to determine a plurality of backbone features 110B characterizing a backbone of the parental protein or a segment thereof.

Specifically, in some embodiments, backbone feature extractor 110 may be configured to determine coordinates 110A of one or more backbone atoms of the parental protein, wherein the backbone atoms include at least one of: a nitrogen (N) atom, a carbon (C) atom, an oxygen (O) atom, an alpha carbon (Cα) atom, and a virtual beta carbon (Cβ).

Backbone feature extractor 110 may be further configured to use coordinates 110A to construct a graph representation of the backbone (e.g., graph representation 110B′). Graph representation 110B′ may have nodes representing amino acids of the parental protein, and edges defined by the backbone features (e.g., features 110B), said backbone features being formed based on at least one of: interatomic distances related to adjacent nodes; characteristics of relative atomic frame orientations and rotations between adjacent nodes; backbone dihedral angles in the adjacent nodes.

In some particular embodiments, extractor 110 may be configured to read structural data 10A; normalize residue numbering (resSeq), chain IDs, insertion codes; resolve altLocs by rule (e.g., highest occupancy). Extractor 110 may then remove solvent and non backbone atoms, retaining N, Cα, C, O (and may compute virtual Cβ if not present). Extractor 110 may be further configured to construct, for each residue i, a local rigid body frame (e.g., using the N—Cα-C triad); compute backbone dihedral angles {φ, ψ, ω} and, where required, χ side chain proxy angles for Cβ placement. Extractor 110 may be further configured to compute edge and node features (e.g., features 110B) for generating graph representation 110B′. Node features (e.g., of features 110B) for residue i may include, e.g.: 3D position of Cα (and frame axes), residue index, secondary structure class (helix/sheet/coil), mask bits (e.g., “frozen/immutable” to preserve antigenic surfaces), and confidence scores. Edge features (e.g., of features 110B) for residue pair (i, j) may include: interatomic distances (Cα-Cα, Cβ-Cβ, N—O, etc.), relative frame orientations/rotations (e.g., quaternion, rotation matrix, or axis angle), sequence separation (|i-j|), contact type, and geodesic distances along the backbone. In embodiments, the extractor 110 may compute a distance/orientation encoding compatible with encoder-decoder based message passing neural network 120 input (network 120 is described further below). In some embodiments, extractor 110 may be further configured to produce a graph representation 110B′ with nodes corresponding to residues and edges defined by computed backbone features 110B; and pack node/edge features of features 110B into tensor arrays (fixed or padded lengths) for batch processing by MPNN 120 inference engine.

In some embodiments, system 100 may further include encoder-decoder-based message passing neural network (MPNN) 120.

In some embodiments, MPNN 120 may represent a deep learning-based protein sequence design model using a graph neural network with message passing and an encoder-decoder structure. It may be configured to infer per-position amino acid probabilities given a protein backbone. E.g., in some non-exclusive embodiments, module 120 may implement or may be configured similarly to ProteinMPNN architecture, as known in the art.

Specifically, in some embodiments, MPNN 120 may be configured to perform preprocessing of graph representation 110B′. E.g., in some embodiments, MPNN 120 may be configured to normalize node and edge features (distances, orientations, dihedral angles, e.g., features 110B); apply masks for non-designable positions (e.g., antibody-binding footprints); and dissociate nodes of representation 110B′ from the amino acids of the parental protein.

In some embodiments, MPNN 120 may be further configured to apply a message-passing algorithm to encode the pre-processed graph representation. E.g., MPNN 120 may use the structure of the message passing neural network to propagate information across nodes and edges, wherein each node may aggregate messages from neighbors based on edge features (distances, orientations). MPNN 120 may be further configured to use encoder layers to compute an encoded graph representation comprising latent information about the structure of the backbone. In some embodiments, said message-passing algorithm may have messages constructed by applying a pretrained multilayer perception (MLP) model on each pair of adjacent nodes and edges therebetween.

MPNN 120 may be further configured to apply a decoding algorithm (e.g., autoregressive decoder) on the encoded graph representation, to predict, per each node and each amino acid (e.g., iteratively), a probability of a respective node to be associated with a respective amino acid. Decoding order can be randomized or order-agnostic, enabling flexible design scenarios (e.g., fixing certain positions, symmetric design). For multi-chain or symmetric designs of MPNN 120, logits for tied positions may be averaged to enforce constraints. Thereby, MPNN 120 may predict the probability for a specific amino acid to be located at the specific position of the backbone. Said probability may be provided, e.g., as logit matrix or normalized probabilities for each residue position (e.g., probability matrix 120A for amino acid identity at each position).

In some embodiments, MPNN 120 may be trained using a large dataset of experimentally determined protein structures, such as high-resolution entries from the Protein Data Bank (PDB), optionally clustered to reduce redundancy. The training objective is to predict amino acid identities for each backbone position given structural features, including interatomic distances (N, Cα, C, O, and virtual Cβ), relative frame orientations, and backbone dihedral angles. As described above, the model may employ an encoder-decoder architecture with multiple message-passing layers to propagate structural context across graph nodes and edges. To improve robustness, Gaussian noise may be added to backbone coordinates during training, enabling the model (network 120) to generalize to predicted structures (e.g., AlphaFold models). The decoding process may be autoregressive and order-agnostic, allowing flexible inference scenarios such as fixing certain positions or enforcing symmetry. Performance can be optimized by sampling decoding orders randomly during training and by incorporating edge updates in addition to node updates. The model (network 120) may be trained to minimize categorical cross-entropy loss over amino acid predictions, and inference diversity can be controlled by adjusting sampling temperature during sequence generation.

In some embodiments, system 100 may further include Position-Specific Scoring Matrix (PSSM) generator 130. In some embodiments, PSSM generator 130 may be configured to define, based on the predicted probability (e.g., probability matrix 120A), a position-specific scoring matrix (PSSM) 130A characterizing, per each position in the original polypeptide chain, amino acid alternatives 130B.

The section below provides certain clarifications regarding PSSMs (such as PSSM 130A) and their application in conventional methods.

A “position-specific scoring matrix” (PSSM), also known in the art as position weight matrix (PWM), or a position-specific weight matrix (PSWM), is a commonly used representation of recurring patterns in biological sequences, based on the frequency of appearance of a character (monomer; amino acid; nucleic acid etc.) in a given position along the sequence. Thus, PSSM may represent the log-likelihood of observing mutations to any of the 20 amino acids at each position. PSSMs are often derived from a set of aligned sequences that are thought to be structurally and functionally related and have become widely used in many software tools for computational motif discovery. In the context of amino acid sequences, a PSSM is a type of scoring matrix used in protein BLAST searches in which amino acid substitution scores are given separately for each position in a protein multiple sequence alignment. Thus, a Tyr-Trp substitution at position A of an alignment may receive a very different score than the same substitution at position B, subject to different levels of amino acid conservation at the two positions. This is, in contrast to position-independent matrices such as the PAM and BLOSUM matrices, in which the Tyr-Trp substitution receives the same score no matter at what position it occurs. PSSM scores may be generally shown as positive or negative integers. Positive scores may indicate that the given amino acid substitution occurs more frequently in the alignment than expected by chance, while negative scores may indicate that the substitution occurs less frequently than expected. Large positive scores may indicate critical functional residues, which may be active site residues or residues required for other intermolecular or intramolecular interactions. PSSMs are conventionally created using Position-Specific Iterative Basic Local Alignment Search Tool (PSI-BLAST), which finds similar protein sequences to a query sequence, and then constructs a PSSM from the resulting alignment. Alternatively, in conventional methods, PSSMs can be retrieved from the National Center for Biotechnology Information Conserved Domains Database (NCBI CDD) database, since each conserved domain is represented by a PSSM that encodes the observed substitutions in the seed alignments. These CD records can be found either by text searching in Entrez Conserved Domains or by using Reverse Position-Specific BLAST (RPS-BLAST), also known as CD-Search, to locate these domains on an input protein sequence. Accordingly, in some embodiments, PSSM generator 130 may be configured to implement one or more of the aforementioned PSSM calculation techniques or incorporate known tools for PSSM 130A calculation.

In the known solutions, a PSSM data file can be in the form of a table of integers, each indicating how evolutionary conserved is any one of the 20 amino acids at any possible position in the sequence of the designed protein. As indicated hereinabove, a positive integer indicates that an amino acid is more probable in the given position than it would have been in a random position in a random protein, and a negative integer indicates that an amino acid is less probable at the given position than it would have been in a random protein. In the known solutions, the PSSM scores are determined according to a combination of the information in the input MSA and general information about amino acid substitutions in nature, as introduced, for example, by the BLOSUM62 matrix.

Conventional methods use the PSSM output of a PSI-BLAST software package to derive a PSSM for both the original MSA and all sub-MSA files. A final PSSM input file may include the relevant lines, such as from each PSSM file. For sequence positions that represent a secondary structure, relevant lines are copied from the PSSM. For each loop, relevant lines are copied from the PSSM derived from the sub-MSA file representing that loop. Thus, in the known solutions, a final PSSM input file is a quantitative representation of the sequence data, which is incorporated in the structural calculations.

Accordingly, in some embodiments, PSSM Generator 130 may convert probability matrix output from MPNN 120 (matrix 120A) into PSSM 130A by logarithmically normalizing the predicted probabilities of matrix 120A (e.g., using logarithmic or log-odds scaling); averaging the normalized probabilities over a plurality of independent initializations, to account for stochastic variations; and defining the PSSM based on the averaged normalized probabilities. Generator 130 may be further configured to apply design constraints such as immutable positions or allowed amino acid sets. The resulting PSSM (e.g., PSSM 130A) may be expressed as a table of integers or floating-point values, where positive scores indicate substitutions more likely than random expectation and negative scores indicate less likely substitutions. PSSM 130A may serve as a quantitative representation of amino acid preferences at each position and is used by the sequence design module to guide combinatorial generation of candidate sequences under structural and functional constraints.

In some embodiments, system 100 may further include sequence design module 140. Sequence design module 140 may be configured to receive, as input, PSSM 130A. In some embodiments, module 140 may be further configured to receive, as additional input, parental sequence 10A′ of amino acids (e.g., may be derived from structural data 10A).

Sequence design module 140 may be further configured to combinatorically generate a plurality of designed sequences (e.g., candidate sequences 140A), wherein each of said designed sequences may correspond to a modified polypeptide chain and comprise one or more amino acid substitutions each being one of said amino acid alternatives 130B.

In some embodiments, module 140 may perform the following operations: (i) combinatorial generation: selecting amino acids for each designable position based on PSSM scores (of PSSM 130A), applying acceptance thresholds to ensure substitutions are consistent with predicted stability and structural integrity; (ii) constraint enforcement: preserving immutable positions (e.g., antigenic surfaces) and applying allowed amino acid sets for specific regions (e.g., cysteine handling for disulfide bonds); (iii) diversity control: introduction of controlled randomness or temperature-based sampling to generate sequence diversity while maintaining high-scoring substitutions; (iv) sequence assembly: construction of full-length candidate sequences by combining selected residues for all positions.

In some embodiments, sequence design module 140 may be configured to combinatorically generate sequences 140A under an acceptance threshold based on the stability scoring of PSSM 130A.

In some embodiments, the modified polypeptide chain (of candidate sequences 140A) may include at least six amino acid substitutions relative to said original polypeptide chain.

In some embodiments, system 100 may further include structure modeling module 150. Structure modeling module 150 may be configured to receive, as an input, candidate sequences 140A (with substitutions). In some embodiments, module 150 may be further configured to receive, as an additional input, parental structure 10A″ (e.g., may be derived from structural data 10A).

In some embodiments, structural modeling module 150 may be further configured to thread each of said designed sequences (e.g., sequences 140A) on a template structure of said original polypeptide chain (e.g., parental structure 10A″), to thereby generate a plurality of designed structures (e.g., candidate structures 150A).

In particular, structural modelling module 150 may map each amino acid in a candidate sequence 140A onto the corresponding backbone position of the template structure (parental structure 10A″), preserving backbone coordinates while replacing side-chain identities (this operation is referred herein as “threading”).

In some embodiments, system 100 may further include energy minimization module 160, configured to receive candidate structures 150A.

Energy minimization module 160 may be further configured to compute an initial energy score for each structure 150A using a composite energy function that may include terms for bond lengths, bond angles, backbone dihedral angles, side-chain packing, hydrogen bonding, electrostatics, solvation effects etc. Module 160 may be further configured to perform at least one of the following energy minimization operations (e.g., iteratively adjusting atomic coordinates, e.g., to reduce steric clashes and optimize geometry): bond length optimization, bond angle optimization, backbone dihedral angles optimization, amino acid side-chain packing optimization, rigid-body optimization, and sidechain dihedral angle minimization of the modified polypeptide chain. Module 160 may be further configured to assign a minimized energy score to each structure (candidate structure 150A). In some embodiments, energy minimization module 160 may be further configured to sort structures 150A according to the minimized energy scoring (as determined by subjecting each structure 150A to an energy minimization).

Accordingly, energy minimization module 160 may be configured to output ranked structures 160A (with minimized energy scores).

In some embodiments, system 100 may further include selection and output module 170, configured to receive ranked structures 160A.

Module 170 may be configured to select at least one of said plurality of designed structures (e.g., structures 160A), each corresponding to the respective modified polypeptide chain, based on said minimized energy scoring. Specifically, selection and output module 170 may identify one or more top-performing designs for experimental implementation and prepare sequence data for downstream expression. E.g., module 170 may review energy scores and optionally other metrics (e.g., predicted solubility, aggregation propensity, antibody-binding footprint compliance) and choose at least one candidate design (candidate structure 160A) that meets predefined thresholds or optimization criteria (e.g., lowest energy score, highest predicted stability).

Accordingly, module 170 may be further configured to output final amino acid sequence 160A of the respective modified polypeptide chain for use as sequence data input for providing a protein expression vector (e.g., vector 180A).

In some embodiments, a selected modified polypeptide chain (corresponding to final amino acid sequence 160A) may correspond to designed structure having a minimal value for said minimized energy scoring, wherein said energy minimization may be a global energy minimization.

In some embodiments, system 100 may further include expression vector construction module 180, configured to receive final amino acid sequence 160A. Module 180 may be configured to prepare the selected amino acid sequence (e.g., sequence 160A) for recombinant expression by generating a protein expression vector suitable for use in a chosen expression system (e.g., protein expression vector 180A). In some embodiments, module 180 may be configured to apply known techniques to generate vector 180A, adapted for the selected protein expression system (e.g., system 190).

In some embodiments, system 100 may further include or be connected to protein expression system 190. Protein expression system 190 may be configured to express the modified protein using protein expression vector 180A. Thereby, protein expression system 190 may be configured to produce modified protein 190A.

In some embodiments, protein expression system 190 may include any suitable recombinant expression platform, such as: prokaryotic systems (e.g., Escherichia coli strains such as BL21(DE3), SHuffle® T7); yeast systems (e.g., Pichia pastoris, Saccharomyces cerevisiae); mammalian systems (e.g., HEK293, CHO cells); etc.

Expression system 190 may operate by introducing the prepared expression vector into the host cells (or cell-free system), enabling transcription of the codon-optimized gene and translation into the modified protein. System 190 may include standard components such as promoters, ribosome binding sites, and selectable markers to ensure efficient expression. After induction under appropriate conditions (e.g., IPTG for E. coli), the target modified protein is produced and can be purified using conventional methods (e.g., affinity chromatography).

In some embodiments, in result of the processing described above, the substitutions (of candidate sequences 140A) may improve the stability of the modified protein 190A relative to the parental protein (defined by structural data 10A), as determined by at least one of: a thermal denaturation temperature of the modified protein being equal or higher than a thermal denaturation temperature of the parental protein; a solubility of the modified protein being equal or higher than a solubility of the parental protein; a degree of misfolding of the modified protein being equal or lower than a degree of misfolding of the parental protein; a half-life of the modified protein being equal or longer than a half-life of the parental protein; a specific activity of the modified protein being equal or higher than a specific activity of the parental protein; and a recombinant expression level of the modified protein being equal or higher than a recombinant expression level of the parental protein.

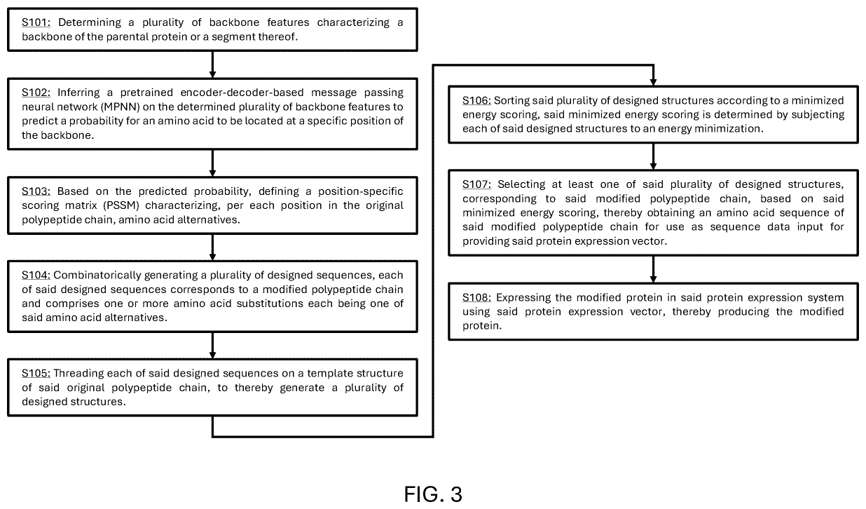

Referring now to FIG. 3, a flow diagram is presented, depicting a method for producing a modified protein, by at least one processor (e.g., processor 2 of FIG. 1), according to some embodiments.

As shown in step S101, said at least one processor (e.g., such as processor 2 of FIG. 1), may determine a plurality of backbone features (e.g., features 110B, as shown in FIG. 2) characterizing a backbone of the parental protein or a segment thereof. Step S101 may be carried out, e.g., by backbone feature extractor (as described with reference to FIG. 2).

As shown in step S102, said at least one processor (e.g., such as processor 2 of FIG. 1), may infer a pretrained encoder-decoder-based message passing neural network (MPNN) (e.g., MPNN 120, as shown in FIG. 2) on the determined plurality of backbone features (e.g., features 110B, as shown in FIG. 2) to predict a probability (e.g. probability matrix 120A, as shown in FIG. 2) for an amino acid to be located at a specific position of the backbone. Step S102 may be carried out, e.g., by MPNN 120 (as described with reference to FIG. 2).

As shown in step S103, said at least one processor (e.g., such as processor 2 of FIG. 1), may define, based on the predicted probability (e.g., probability matrix 120A, as shown in FIG. 2), a position-specific scoring matrix (PSSM) (e.g., PSSM 130A, as shown in FIG. 2) characterizing, per each position in the original polypeptide chain (e.g., defined by structural data 10A, as shown in FIG. 2), amino acid alternatives (e.g., amino acid alternatives 130B, as shown in FIG. 2). Step S103 may be carried out, e.g., by PSSM generator 130 (as described with reference to FIG. 2).

As shown in step S104, said at least one processor (e.g., such as processor 2 of FIG. 1), may combinatorically generate a plurality of designed sequences (e.g., candidate sequences 140A, as shown in FIG. 2), each of said designed sequences corresponds to a modified polypeptide chain and comprises one or more amino acid substitutions each being one of said amino acid alternatives (e.g., amino acid alternatives 130B, as shown in FIG. 2). Step S104 may be carried out, e.g., by sequence design module 140 (as described with reference to FIG. 2).

As shown in step S105, said at least one processor (e.g., such as processor 2 of FIG. 1), may thread (e.g., map) each of said designed sequences (e.g., candidate sequences 140A, as shown in FIG. 2) on a template structure (e.g., parental structure 10A″, as shown in FIG. 2) of said original polypeptide chain, to thereby generate a plurality of designed structures (e.g., candidate structures 150A, as shown in FIG. 2). Step S105 may be carried out, e.g., by structure modeling module 150 (as described with reference to FIG. 2).

As shown in step S106, said at least one processor (e.g., such as processor 2 of FIG. 1), may sort said plurality of designed structures (e.g., candidate structures 150A, as shown in FIG. 2) according to a minimized energy scoring (e.g., to output ranked structures 160A), said minimized energy scoring is determined by subjecting each of said designed structures (e.g., candidate structures 150A, as shown in FIG. 2) to an energy minimization. Step S106 may be carried out, e.g., by energy minimization module 160 (as described with reference to FIG. 2).

As shown in step S107, said at least one processor (e.g., such as processor 2 of FIG. 1), may select at least one of said plurality of designed structures (e.g., candidate structures 150A, as shown in FIG. 2), corresponding to said modified polypeptide chain, based on said minimized energy scoring (e.g., according to ranked structures 160A, as shown in FIG. 2), thereby obtaining an amino acid sequence (e.g., final amino acid sequence 160A, as shown in FIG. 2) of said modified polypeptide chain for use as sequence data input for providing said protein expression vector (e.g., protein expression vector 180A, as shown in FIG. 2). Step S107 may be carried out, e.g., by selection and output module 170 and expression vector construction module 180 (as described with reference to FIG. 2).

As shown in step S108, said at least one processor (e.g., such as processor 2 of FIG. 1), may express the modified protein in said protein expression system (e.g., protein expression system 190, as shown in FIG. 2) using said protein expression vector (e.g., protein expression vector 180A, as shown in FIG. 2), thereby producing the modified protein (e.g., modified protein 190A). Step S108 may be carried out, e.g., by protein expression system 190 (as described with reference to FIG. 2).

The claimed invention has undergone research evaluation and demonstrated high efficiency in practical applications. Specifically, the evaluation was directed to improving the stability and recombinant expression yield of the Plasmodium vivax Reticulocyte Binding Protein 2b (PvRBP2b), a diagnostically relevant antigen, while preserving its antigenic profile for recognition by naturally acquired human monoclonal antibodies. This evaluation included computational design using the claimed system and method, followed by experimental expression in E. coli and biophysical characterization of the resulting protein variants.

The evaluation demonstrated that stabilized PvRBP2b designs generated by the claimed approach exhibited up to 11-fold higher expression yields compared to the parental protein and improved thermal stability by 8-14° C., as measured by dynamic light scattering and label-free thermal shift analysis. Importantly, the modified proteins retained binding to a panel of human monoclonal antibodies with nanomolar affinities, confirming preservation of immunologically critical surfaces. These results validate the technical feasibility and advantages of the claimed concept.

However, it shall be understood that this specific purpose of evaluation is provided solely as a non-exclusive example illustrating the effectiveness of the claimed invention. The present invention is not limited in any way to the particular target protein used in the evaluation or to the purpose of such modification. Rather, the disclosed system and method are broadly applicable to any protein requiring sequence optimization for enhanced stability, expression, or other desirable properties, including but not limited to enzymes, therapeutic proteins, and vaccine immunogens.

Plasmodium vivax is emerging as the most prevalent species causing malaria outside Africa. Most P. vivax infections are relapsed due to the reactivation of the dormant liver stage parasites (hypnozoites). Hypnozoites are a major reservoir for transmission but undetectable by commercial diagnostic tests. Antibodies against P. vivax Reticulocyte Binding Protein 2b (PvRBP2b) are among the most reliable serological biomarkers for recent infections in the prior nine months and provide indirect biomarkers for risk of relapse The research was thus aimed at designing stabilized variants of PvRBP2b, under stringent conditions of minimally perturbing the solvent-accessible surfaces to maintain its antigenicity profile. Furthermore, for some of the designs, due to limited diversity of natural PvRBP2b homologs, it is suggested herein to combine AI-based sequence design tools and atomistic design calculations (as described with reference to FIG. 2). The best, bearing 19 core mutations relative to PvRBP2b, expressed up to 11 mg per L, and had 14° C. higher thermal tolerance than the parental protein. Critically, the stabilized designs retained binding to naturally acquired human monoclonal antibodies with nanomolar affinities, suggesting that the immunologically competent surfaces were retained as was confirmed by crystallographic analyses. Using longitudinal clinical cohorts from malaria endemic regions of Thailand, Brazil and the Solomon Islands, the research demonstrated that antibody responses against the designs are highly correlated with those against the parental protein and can classify individuals as recently infected with P. vivax. This efficient computational stability design methodology can be used to enhance the biophysical properties of other recalcitrant proteins for use as diagnostics or vaccine immunogens.

Plasmodium vivax is a major cause of relapsing human malaria-causing species and remains a key obstacle to malaria elimination. Antibody responses against the P. vivax RBP2b protein are among the leading serological biomarkers for recent infection, indirectly identifying individuals with a high likelihood of relapse and can be used to determine whether to administer antimalarial treatment. The present disclosure describes a streamlined computational methodology to design variants of PvRBP2b for increased production yields and greater thermal stability while retaining immunogenicity to naturally acquired human monoclonal antibodies. This method has broad potential to quickly overcome barriers to the implementation of economical and resilient diagnostics and vaccine immunogens for many infectious diseases.

Plasmodium vivax remains a key obstacle to malaria elimination. P. vivax is the most widely distributed parasite with estimated 4-7 million annual cases in Asia, Oceania, and the Americas. A major challenge to P. vivax elimination is its dormant form, the hypnozoite, which evades existing control interventions to cause relapsing infections. By doing so, the parasite can maintain ‘hidden’ low-density infections that sustain ongoing transmission.

Due to dormancy, individuals carrying hypnozoites are clinically silent yet are a reservoir for transmission. A recent in-development diagnostic is currently able to detect infections within the prior 9-months with 80 % sensitivity and 80% specificity. This diagnostic comprises a panel of eight P. vivax proteins that serve as serological exposure markers capable of classifying individuals with recent P. vivax infections who have a high likelihood of harboring hypnozoites and should be targeted with anti-hypnozoite therapy. Among these markers, the P. vivax reticulocyte binding protein 2b (PvRBP2b), a member of the PvRBP family of protein adhesins, produces the top prediction for dormant P. vivax infection. PvRBP2b binds to human Transferrin Receptor 1 (TfR1) to mediate entry into reticulocytes. In addition, it is a target of naturally acquired immunity, and several longitudinal cohort studies show that antibodies to PvRBP2b are correlated with clinical protection. Naturally acquired human monoclonal antibodies against PvRBP2b functionally inhibit its binding to reticulocytes and block the interaction between PvRBP2b and TfR1.

PvRBP2b can be produced in recombinant microbial systems resulting in a well-folded and functional protein. Nevertheless, its expression yields and stability are low, limiting its development as a serological marker for diagnostic tests, particularly for use in malaria endemic countries, which are the main targets for elimination efforts. Several computational and experimental approaches have been developed to address marginal protein stability and low expressibility. Typically, stabilizing mutations are introduced in solvent-exposed positions because those are less likely to compromise protein foldability. In the case of PvRBP2b, however, structural analyses indicated that >48% of the solvent-accessible surface is targeted by naturally acquired human antibodies from previously infected individuals. To serve as a useful serological marker, the designed antigen must exhibit nearly the same antigenic profile as parental PvRBP2b, dictating that much of the surface should be free of mutations, and that the natural protein backbone conformation must be carefully maintained. Therefore, the design strategy should mostly introduce core mutations without, however, perturbing the backbone structure.

Introducing many stabilizing core mutations while mostly maintaining the protein surface demands very high accuracy in design. Due to these stringent requirements, the research started with trying to apply known natural homolog-based protein stability-design methods.

Known natural homolog-based protein stability-design methods (e.g., PROSS) have been successfully applied to dozens of different enzymes, binders, and vaccine immunogens, including P. falciparum Rh5 (PfRh5), the leading vaccine candidate against P. falciparum blood stages. In many cases, including PfRh5, designs exhibited much improved thermal and kinetic stability and expressibility without impacting protein activity, and the stabilized PfRh5 design has recently entered Phase II clinical trials in West Africa (31). Although the previous successful applications of such known methods are encouraging, they have not been applied to design diagnostic antigens in which surface mutations were strongly limited. A further complication is that such known methods often rely on a multiple-sequence alignment of homologs to restrict atomistic design choices to amino acid identities that are commonly observed in the natural diversity. This aspect of the their methodology is critical to its ability to design dozens of simultaneous mutations in large and complex proteins without leading to aggregation, misfolding, and loss of function. Due to the limited natural diversity of Plasmodia species, however, the research only detected a few dozen PvRBP2b homologs in sequence databases. Therefore, it is suggested herein to combine the recent structure- and AI-based sequence design approach (e.g., similar to the one applied by ProteinMPNN) with the atomistic design calculations (e.g., similar to the ones applied by PROSS) thus circumventing the requirement for a deep multiple-sequence alignment.

Using the suggested combined workflow, during the research, stabilized PvRBP2b designs with their immunogenic properties retained were successfully generated. The best of the nine experimentally tested embodiments of the present solution design exhibited higher yields and increased thermal stability while maintaining binding to all naturally acquired human monoclonal antibodies in the research panel. Using a multiplex approach, the obtained results show that the PvRBP2b designs maintained the same sensitivity and specificity as the parental protein in serological tests.

Aspects of the research conducted during the development of the solution suggested herein are described below.

Computational design. PvRBP2b is a 326 kDa protein with a putative red blood cell binding domain and a C-terminal transmembrane region. The surface of the N-terminal domain of the natural protein PvRBP2b (residues 169 to 470, PvRBP2b₁₆₉₋₄₇₀) is a mostly positively charged α-helical protein comprising ten α-helices and two very short antiparallel β-sheets. The crystal structure of PvRBP2b₁₆₉₋₄₇₀ has two disulfide bonds. All natural fragments with the N-terminal domain bound reticulocytes (PvRBP2b₁₆₁₋₁₄₅₄, PvRBP2b₁₆₁₋₉₆₉, PvRBP2b₁₆₉₋₈₁₃ and PvRBP2b₁₆₉₋₆₅₂) whereas their corresponding fragments without the domain did not (PvRBP2b₄₇₄₋₁₄₅₄ and PvRBP2b₄₇₄₋₉₆₉). It has been previously shown that PvRBP2b₁₆₉₋₄₇₀ can achieve equal sensitivity and specificity at predicting recent P. vivax exposure compared to its larger counterpart PvRBP2b₁₆₁₋₁₄₅₄. Therefore, the research included designing stabilized variants using PvRBP2b₁₆₉₋₄₇₀ as the template sequence, which is further referred herein as the parental construct (FIG. 4).

The conventional stability design algorithms start by searching nonredundant sequence database for homologs and aligning them. The resulting multiple-sequence alignment (MSA) is then used to generate a statistical model of substitutions at each amino acid position (position-specific scoring matrix; PSSM, such as PSSM 130A, shown in FIG. 2) to guide Rosetta atomistic design choices. The atomistic design step uses a physics-based energy function, dominated by van der Waals interactions, electrostatics, hydrogen bonding, and solvation, to optimize the native-state stability. During design calculations, mutations that are rare according to the PSSM are disallowed, and the remaining mutations are weighted according to their occurrence. Like many surface antigens from Plasmodia, however, PvRBP2b exhibits significant homology to only a few dozen sequences in the nonredundant sequence database, and it was reasoned that a conventional PSSM computed from sequence homologs might not be a sufficiently accurate guide for design calculations.

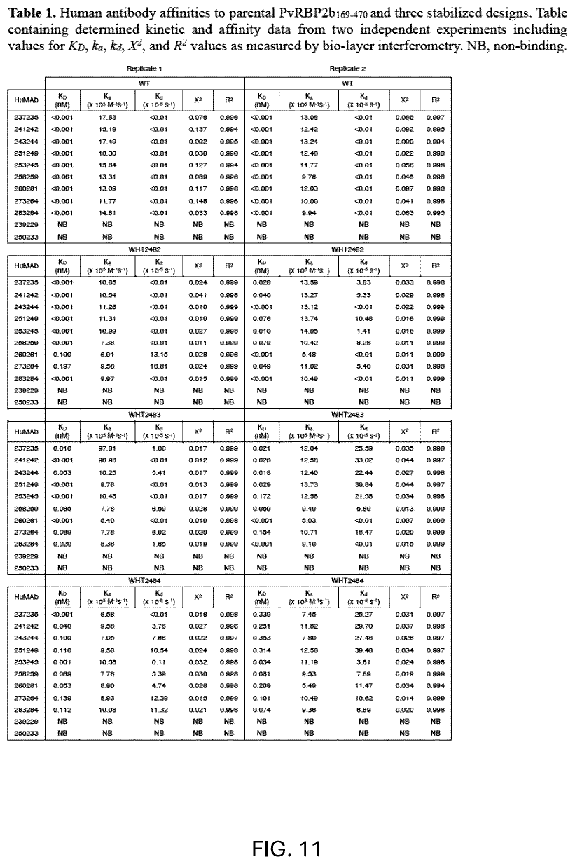

To address the limited diversity of PvRBP2b homologs in nature, the research included experimenting with a different strategy for generating a PSSM using known AI-based sequence design tools, e.g., the ones using a graph neural network architecture trained on structures and sequences observed in the Protein Data Bank (PDB) to design new sequences given a protein backbone without relying on information from homologs (e.g., similar to the operation of MPNN 120, described with reference to FIG. 2). These approaches was successfully applied to de novo designs, the majority of which were based on small α-helical structures. Due to the high α-helix content of PvRBP2b₁₆₉₋₄₇₀ (79%), it was hypothesized that such known AI tools may make accurate predictions in this protein. However, previous applications of them to natural proteins (which are typically larger and topologically more complex than de novo designed ones) showed lower success rates than known natural homolog-based protein stability-design methods and required significant experimental screening, which would be impractical in the case of a complex protein such as PvRBP2b. To address this potential problem, rather than designing PvRBP2b variants entirely by such AI tools, the research used the aspects of such tools to generate a PSSM (e.g., PSSM 130A, as shown in FIG. 2) as a replacement for the natural homolog-based PSSM in the natural homolog-based protein stability-design workflows. The suggested implementation therefore combines AI tools, in particular, a pretrained encoder-decoder-based message passing neural network (MPNN) with atomistic design calculations and eliminates the requirement of a deep MSA of homologs (FIG. 4). In all calculations, the research excluded positions involved in antibody interfaces from the designs to maintain the parental antigenic profile. The research included designing six variants with the standard phylogenetic strategy (encoding 20-37 mutations from wild type; designs WHT2476 to WHT2481) and three with the MPNN-based PSSMs that exhibited fewer mutations (17-26; designs WHT2482, WHT2483 and WHT2484) (FIG. 4, right-hand side: 6 PROSS designs and 3 PROSS designs based on ProteinMPNN PSSM, respectively; and Table 1, shown in FIG. 11).

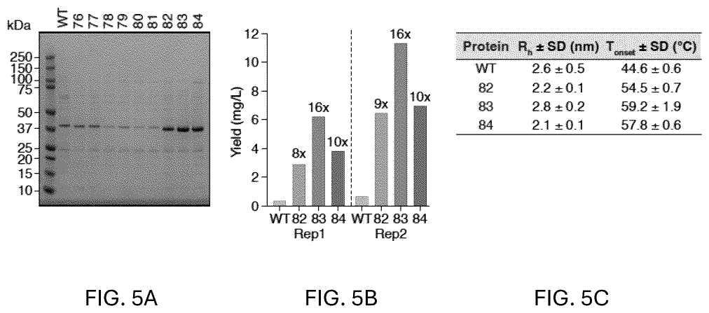

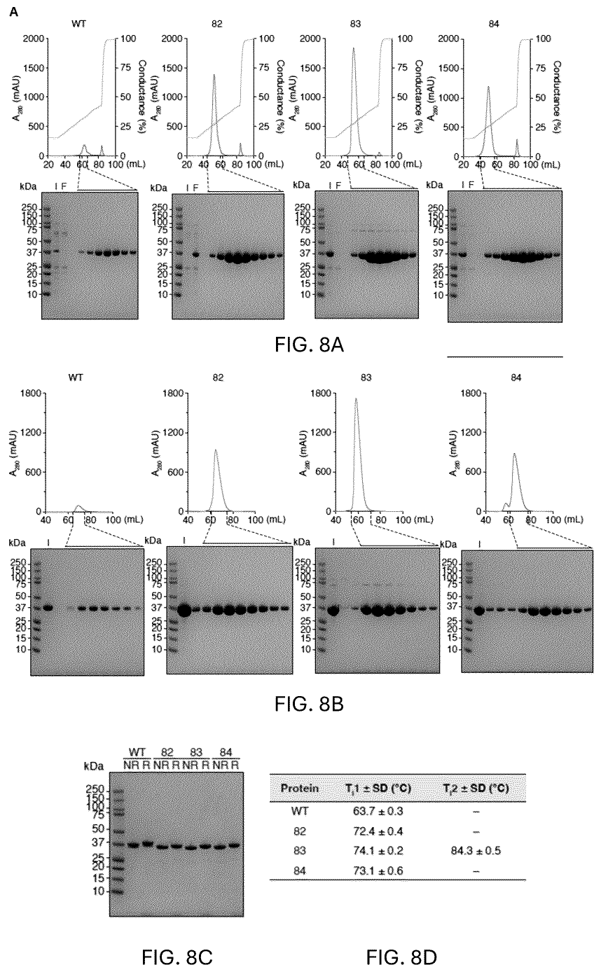

The results demonstrated that the suggested designs have improved yields and increased thermal stability. The research included expressing parental PvRBP2b₁₆₉₋₄₇₀ and the nine designs in Escherichia coli and used Ni-NTA affinity chromatography as the first purification step. SDS-PAGE analyses of purified proteins after Ni-NTA affinity chromatography showed that all three PvRBP2b₁₆₉₋₄₇₀ designs (WHT2482, WHT2483 and WHT2484) made using the suggested method had increased yields relative to the parental construct (FIG. 5A), whereas only two designs (WHT2476 and WHT2477) from the standard natural homolog-based protein stability-design workflow expressed as well as the parent. For the three designs (WHT2482, WHT2483 and WHT2484) made using the suggested method, the research proceeded with two additional purification steps using cation exchange and size exclusion chromatography (FIG. 5B and FIG. 8A-C). After the three-step purification process, WHT2482, WHT2483 and WHT2484 showed increased protein yields up to 8, 16 and 10-fold respectively, compared to parental PvRBP2b₁₆₉₋₄₇₀ (FIG. 5B). WHT2482, WHT2483 and WHT2484 had final yields up to 6, 11 and 7 mg per liter of E. coli culture respectively compared to 0.7 mg per liter for the parent (FIG. 5B).

Dynamic light scattering measurements showed that the three designs displayed significantly higher temperature of aggregation onset (Tₒₙₛₑₜ) values of 54 to 59° C. compared to 45° C. for the parental protein (FIG. 5C), translating to an improvement of 9 to 14° C. The research also included conducting thermal stability measurements using a label-free differential scanning fluorimeter and observing that the designs showed higher thermal stability as described by the inflection temperature (Tᵢ) by 8 to 10° C. compared to parental PvRBP2b₁₆₉₋₄₇₀ (FIG. 8D).

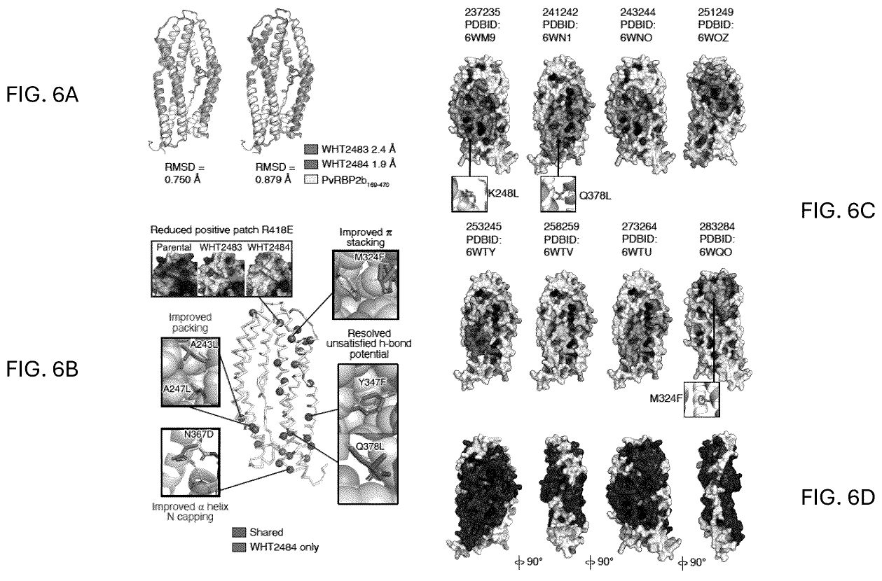

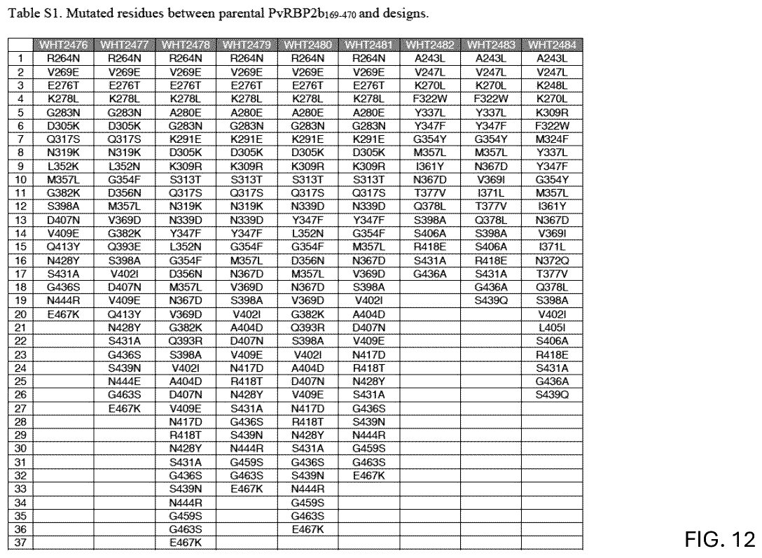

It was established that PvRBP2b structural fold is retained for stabilized designs. To determine whether the designs retained the structure of the parental protein, the research included determining the crystal structures of WHT2483 and WHT2484 (19 and 26 mutations, respectively) to 2.4 Å and 1.9 Å resolution respectively (FIG. 6A and Table S1, shown in FIG. 12). An overlay of the structural coordinates of parental PvRBP2b₁₆₉₋₄₇₀ (PDB ID 5W53) with WHT2483 and WHT2484 showed very similar structural scaffolds with RMSDs of 0.750 Å and 0.879 Å respectively (FIG. 6A). The biggest divergence in the backbones occurred at the C-terminal portions of the α5 and α7 helices with up to 1.7 Å difference in position at His464 in the α7 helix in WHT2484.

To assess the reasons for the higher bacterial expression levels and improved thermal stability of the design, the research included comparing their structure to that of the parental PvRBP2b₁₆₉₋₄₇₀. This comparison showed several of the hallmarks expected for stabilizing mutations including a reduction in the size of homogenously charged surface patches, improved core packing, improved π stacking and removal of buried polar residues that exhibit unsatisfied hydrogen bond donors or acceptors (FIG. 6B and Table S1, shown in FIG. 12). Most of these changes affect the solvent inaccessible interior of the proteins, which are not directly involved in antibody recognition.

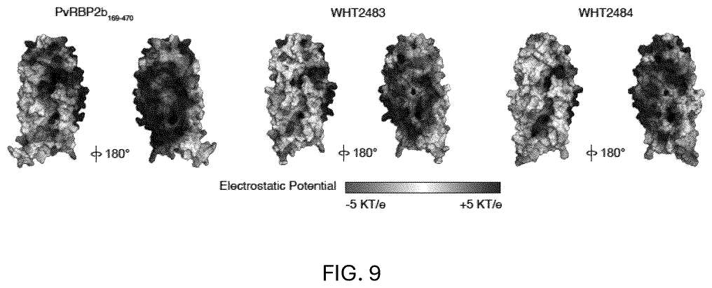

The main surface change is the reduction in positive charge in a patch on both WHT2483 and WHT2484 compared to the parental construct due to the introduction of the charge-swap mutation Arg418Glu (FIG. 6B). This is reflected in the overall reduction in the calculated isoelectric point (pI) from 9.18 for parent to 8.91 and 8.82 for WHT2483 and WHT2484, respectively. Apart from this region, however, the charge patterns remained similar (FIG. 9).

It was further established that PvRBP2b₁₆₉₋₄₇₀ stabilized designs retain human monoclonal antibody recognition.

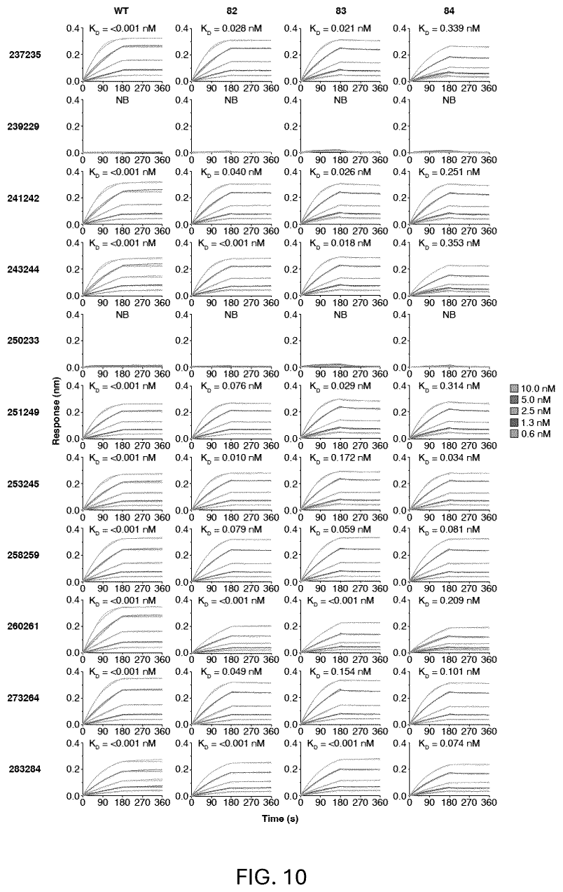

To ensure that the stabilized designs maintained the epitopes for human antibody recognition, the research included determining the binding kinetics and affinities of the interaction between human monoclonal antibodies (mAbs) and WHT2482, WHT2483 and WHT2484 using bio-layer interferometry (BLI) (Table 1, shown in FIG. 11). These nine mAbs were chosen because they bind parental PvRBP2b₁₆₉₋₄₇₀ with picomolar affinity, and high-resolution crystal structures of their antigen-binding fragments (Fab) with this domain were obtained (FIG. 6C). In addition, 48% of the antigen is engaged for interaction by these mAbs (FIG. 6D). The results showed that WHT2482, WHT2483 and WHT2484 bound all nine human antibodies with affinity in the low nanomolar range (Table 1, shown in FIG. 11, and FIG. 10). While the affinities were not uniformly as high compared to the parental PvRBP2b₁₆₉₋₄₇₀, it was clear that the three stabilized designs retained recognition to a panel of naturally acquired human mAbs.

Comparison of the binding footprints of the human antibody panel with the mutations introduced in WHT2483 and WHT2484 reveals that the binding surfaces are largely conserved, with few of the introduced mutations being within the antibody bound surface of parental PvRBP2b₁₆₉₋₄₇₀ (FIG. 6C). There are three introduced mutations which fall within the antibody binding footprints, these are Lys248Leu and Met324Phe which are present in WHT2484, as well as Gln378Leu which is present in both WHT2483 and WHT2484 (FIG. 6C). These mutations fall within the binding footprints of human mAbs 237235, 283284 and 241242, respectively. Despite the presence of these mutations, these human mAbs still bound with nanomolar affinities to the stabilized designs (Table 1, as shown in FIG. 11), showing recognition was not abolished with the introduction of the three mutations.

It was further established that stabilized designs function as reliable serological markers for recent P. vivax infection.

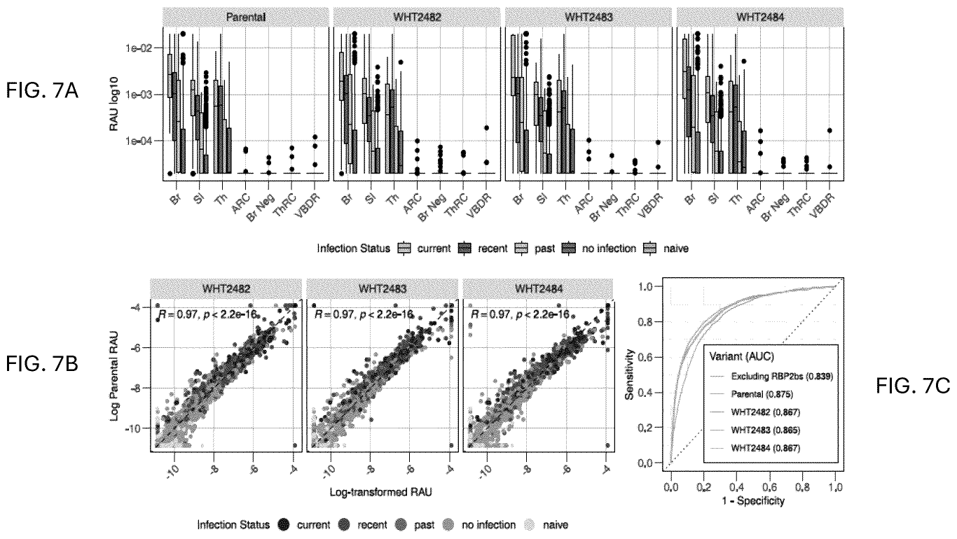

To ensure that the stabilized designs maintain recognition of human polyclonal antibody responses, the research included using a multiplexed Luminex assay to measure IgG antibody responses against the parental construct and WHT2482, WHT2483 and WHT2484 in individuals from malaria-endemic regions of Brazil, Thailand and the Solomon Islands (FIG. 7A). Like the IgG response against the parental construct, design-specific IgG responses were highest in individuals with current and recent (prior nine months) P. vivax infections, declining with increasing time since the last detected P. vivax infection (FIG. 7A). The variant-specific IgG responses were highly correlated with parental PvRBP2b₁₆₉₋₄₇₀ (r=0.97, p<0.001) (FIG. 7B). To classify recent P. vivax exposure, we previously demonstrated that combinations of IgG responses against multiple P. vivax proteins provides better accuracy than using responses to single proteins. The research therefore included testing the ability of the designs to replace the parental construct in a Random Forest classification algorithm and measured the impact on performance. Comparable sensitivity, specificity and area under the curve values were obtained for the parent and the designs as demonstrated by the receiver operator characteristic curves (FIG. 7C). An alternative option for diagnostic development is to replace PvRBP2b with a different P. vivax antigen which is more stable. However, the research demonstrated substantial loss of performance when no PvRBP2b antigen was included, showing the importance of PvRBP2b in the diagnostic classification (FIG. 7C).

As discussed herein, the research included designing mutants of the leading P. vivax diagnostic candidate PvRBP2b that exhibit improved expression yields and thermal stability, while retaining antigenicity and diagnostic classification performance. This generated an improved recombinant antigen that may be more economical to produce and thermally stable for delivery and long-term storage. Cost of production and refrigeration-free transport are two critical determinants for the feasibility of diagnostics intended for use in developing countries, and our designs may aid efforts to implement PvRBP2b in endemic regions. PvRBP2b belongs to the PvRBP family of proteins which are homologous to the P. falciparum Reticulocyte-binding Homolog (PfRh) family. Several members of the PvRBP and PfRh family are known to be important in red blood cell invasion and their human red blood cell receptors have been identified; PvRBP2b binds to TfR1, PvRBP2a binds to CD98, PfRh5 binds to basigin and PfRh4 binds to Complement Receptor 1. The crystal structure of the N-terminal domain of PvRBP2b closely resembles the structures of the homologous domains of PvRBP2a and PfRh5. While several PvRBP and PfRh proteins have been successfully made in different expression systems, there exist only a handful of high-resolution crystal structures of these important parasite adhesins as described above. Given that the design approach we developed and validated generates stable designs while minimally perturbing antigenic surfaces, it may be applied to AI-based model structures of these adhesins to enable crystallographic analysis including of complexes. Such structures are critical for developing diagnostics and vaccine immunogens.

This work builds upon our prior research demonstrating that PvRBP2b is currently the most important P. vivax antigen in the proposed serological biomarker panel. However, the present invention shall not be considered limited in this regard. The research further demonstrates the importance of our approach to improve thermal stability and yield, as exclusion of any PvRBP2b protein in the classification algorithm resulted in decreases in performance to a level that likely deteriorates the reliability of the serological test and treatment approach. Whilst there may be approaches to overcome this drop in performance, such as repeated rounds of serological screening or better coverage, these are likely to increase the overall cost of the intervention and burden to patients and medical staff.

The approach used here is also highly applicable for the development of other serological biomarkers which require increased stability while maintaining immunogenicity. Notably, the three designs made according to the suggested method were all stabilized and highly expressed relative to the parental protein, demonstrating the high reliability of this design strategy. This approach therefore enables rapid and significant enhancement of stability and expression levels in one test cycle, rather than relying on laborious cycles of design and experimental testing which would be impractical for a 39 kDa protein with multiple different binding partners. It is therefore expected this method to contribute to serological biomarker and vaccine production across a wide range of infectious diseases affecting humanity.

Known natural homolog-based protein stability-design methods have been successfully applied to dozens of challenging proteins of biomedical and technological use. Nevertheless, its reliance on crystallographic structures and diverse sequence homologs has restricted its application. The advent of AI-based structure predictors, such as AlphaFold, has eliminated the need for crystallographic structures in many cases. Now, the research results showed that known AI-based sequence generators, may further eliminate the need for diverse sequence homologs; it should be noted, however, that the reliability of sequence generators in proteins that exhibit a low fraction of secondary-structure content should be carefully examined. Together, these advances in protein modeling and design may expand the reach of computational protein engineering, in principle, to any known protein.

The aspects of the suggested system design that were used during the research are discussed below.

PvRBP2b crystal structures were analysed to identify regions that interact with human antibodies from HDX-MS and crystallography data (PBD ID: 5W53, 6WM9, 6WN1, 6WNO, 6WOZ, 6WTY, 6WTV, 6WTU and 6WQO). Surfaces in these regions were disabled from design (FIGS. 6C and 6D). For designs WHT2476 to WHT2481, the PROSS protocol was applied as known in the art. For designs WHT2482 to WHT2484, the research included replacing the multiple-sequence-alignment-based PSSM by a pseudo-PSSM derived from the statistical representation of parental PvRBP2b₁₆₉₋₄₇₀ by the MPNN. Using the backbone coordinates of parental PvRBP2b₁₆₉₋₄₇₀ (PBD ID: 5W53), the MPNN built a backbone-specific mutation model. Log likelihoods for amino acid substitution at each position were extracted from the model. Probabilities were averaged over 50 independent initializations to account for stochastic variations and rounded to the closest integer. The resulting matrix was then treated as a standard PSSM within the atomistic design workflow, similar to the ones used in known natural homolog-based protein stability-design methods. Subsequent structure-based energy filtering of mutations and design steps were performed similar as in the known natural homolog-based protein stability-design methods for all designs. Final designs were analysed visually and point mutations with low alpha helix propensity were eliminated prior to DNA synthesis.

PvRBP2b₁₆₉₋₄₇₀ and designs cloning and sequencing were further performed. Sequence of PvRBP2b from P. vivax strain Salvador I was obtained from PlasmoDB Database (www.plasmodb.org; accession number: PVX_094255, 2,806 amino acids). Synthetic DNA was codon-optimized for expression in E. coli (Life Technologies). The nucleotide sequence encoding amino acids 169 to 470 of PvRBP2b was cloned into pPROEX HTb vector which included sequences for a N-terminal 6×His-tag followed by a TEV cleavage site. This sequence refers to the parental PvRBP2b₁₆₉₋₄₇₀. Restriction enzyme cloning was used to clone the synthetic DNA fragments (obtained from Twist Biosciences) of PvRBP2b₁₆₉₋₄₇₀ designs into pPROEX HTb vector. All positive plasmids were sequence verified at the WEHI Advanced Genomics Facility.

Expression and purification of PvRBP2b₁₆₉₋₄₇₀ designs are discussed further below.

Parental PvRBP2b₁₆₉₋₄₇₀ was expressed using E. coli strain SHuffle® T7 (New England Biolabs) and Terrific Broth (TB) supplemented with 100 μg/mL of carbenicillin. Flasks containing 1 L of medium were incubated in a Multitron shaker (Infors HT) at 37° C. at 180 rpm. At OD₆₀₀ of around 1.0, IPTG (Astral) was added to the final concentration of 1.0 mM and protein expression was allowed to continue for 20 hours at 16° C. Cells were harvested by centrifugation at 6,000×g, resuspended in freezing buffer containing 50 mM Tris HCl pH 7.5, 500 mM NaCl, 10 % (v/v) glycerol supplemented with cOmplete EDTA-free protease inhibitor cocktail (Roche) and stored at −80° C. until further processing.

For the purification, cell pellet was thawed on ice and resuspended in the freezing buffer supplemented with 0.5 mg/mL of DNase and 1.0 mg/mL of lysozyme (Sigma-Aldrich). Cells were lysed using sonicator Sonopuls UW 3200 (Bandelin) equipped with VS 70 T probe. The obtained crude cell extract was clarified by centrifugation at 30,000×g for 45 minutes at 4° C. The supernatant was loaded onto the 5 mL HisTrap excel column (GE Healthcare) pre-equilibrated with the freezing buffer. Unbound material was removed using 10 column volumes of wash buffer: 20 mM Tris HCl pH 7.5, 500 mM NaCl and 10 mM imidazole. The bound protein was eluted from the column using the same buffer but containing 300 mM imidazole. Eluted fractions were pooled and dialyzed overnight into the dialysis buffer containing 20 mM Tris pH 7.5 and 100 mM NaCl. The resulting protein sample was applied on the 5 mL HiTrap SP HP cation exchange chromatography column (GE Healthcare) pre-equilibrated with the dialysis buffer. Unbound material was removed using 10 column volumes of the buffer. Protein was eluted from the column using a gradient of 20 mM Tris pH 7.5 and 1.0 M NaCl. Collected fractions were analyzed on SDS PAGE and fractions of interest were concentrated using an Amicon Ultra-4 10 kDa molecular weight cut-off concentrator (Millipore) and loaded onto S75 Superdex 16/600 size exclusion column (GE Healthcare) pre-equilibrated with 20 mM HEPES pH 7.5 and 150 mM NaCl. The monodisperse peak fractions containing protein were pooled and concentrated using the same concentrator, flash-frozen in liquid nitrogen and stored at −80° C. Expression and purification of WHT2482, WHT2483 and WHT2484 designs were performed in a similar manner as described above.

Parental PvRBP2b₁₆₉₋₄₇₀ and designs were diluted to 1 mg/mL and transferred to an Aurora 384 well plate for dynamic light scattering measurements in the DynaPro plate reader III (Wyatt Technology). All samples were centrifuged at 17,000×g for 5 min at 4° C. to sediment any precipitate. The plate was sealed, and measurements were performed over a temperature ramp of 25 to 80° C. at a ramp rate of 0.1° C./min, with 4 s acquisition time averaged over 5 acquisitions. The onset model was fit to the data to obtain Tonset values for all constructs. Tₒₙₛₑₜ values are the temperature at which the protein begins to unfold. Data from at least two different batches of protein in triplicate were evaluated using Dynamics software v8.0.0.89.

Thermal shift assays were performed using the Tycho NT.6 (NanoTemper Technologies). Parental PvRBP2b₁₆₉₋₄₇₀ and designs were measured at 10 μM in storage buffer. 10 uL of each sample was transferred into a capillary and measured from 35 to 95° C. using a Tycho NT.6 (Nanotemper). The inflection temperatures of each protein were calculated by the Tycho NT.6 software (1.2.0.750). Technical triplicates were measured in three independent experiments. Data were analyzed using GraphPad Prism.

Crystallization trials were undertaken at the Bio21 Collaborative Crystallization Facility at 20° C. using 96 well sitting drop vapour diffusion plates (Greiner). Crystals were obtained for WHT2483 from a solution containing 5% (v/v) MPD, 10% (w/v) PEG 6000 and 0.1 M HEPES pH 7.5 at 10 mg/mL. Crystals were obtained for WHT2484 using 10% (v/v) Propan-2-ol, 10% (w/v) PEG MME 5000, 0.1 M Na Cacodylate pH 6 at 15 mg/mL. Crystals were flash frozen in liquid nitrogen at 100 K following cryoprotection with 15 to 20% glycerol in reservoir solution. Datasets were collected to 2.4 Å (WHT2483) and 1.9 Å (WHT2484) using the MX2 beamline at the Australian Synchrotron (Melbourne, Victoria). Data were recorded using an Eiger 16M detector (Dectris) and processed using the XDS package. Molecular replacement was undertaken using Phaser. A search model was generated using AlphaFold2 models for WHT2483 and WHT2484 respectively.

The WHT2483 structure was solved in space group P2₁2₁2₁ with two copies in the asymmetric unit while WHT2484 was solved in space group P4₁2₁2 and had a single copy in the asymmetric unit. Refinement through iterative rounds of model building in COOT and refinement in Phenix v1.19.2 generated models with an R_{obs}/R_{free}=19.52/24.53 for WHT2483 and 18.88/22.17 for WHT2484. Structural models were deposited in the PDB under PDB ID 9DZC and 9DZD respectively.

Recombinant human monoclonal antibodies (mAbs) were expressed in Expi293 HEK cells (Life Technologies) maintained in suspension at 37° C. and 8% CO₂. Cells were transfected at a density of 3×10⁶ with equal amounts of heavy and light-chain paired plasmids using polyethyleneimine (PEI, Sigma-Aldrich) at a ratio of 1:03 of the total amount of plasmid to PEI. One day after transfection, valproic acid was added to cultures to a final concentration of 0.025 M. Seven days after transfection, the supernatant was collected by centrifugation and filtered through a 0.22 μm filter. Human mAbs were purified by loading the supernatant onto a 1 mL Protein A HP HiTrap column (GE Healthcare). Columns were equilibrated and washed using Dulbecco's phosphate-buffered saline (DPBS). Human mAbs were eluted using 0.1 mM citric acid pH 3.00 and neutralized with 1 M Tris-HCl pH 9.0. A second purification step was performed by loading Protein A eluate on a Hiload 16/600 Superdex 200 pg gel filtration column (GE Healthcare), which was pre-equilibrated with DPBS. Human mAbs were concentrated using Amicon Ultra-04 5 kDa (Millipore). Antibody concentration was determined by absorbance measurement at 280 nm using a Nanodrop and purity was determined using SDS-PAGE.

Antibody affinities were measured using an Octet RED96 instrument. Assays were performed at 25° C. in solid black 96-well plates agitated at 1000 rpm. The kinetic buffer was composed of PBS 0.1% BSA, 0.05% TWEEN. A 60 s biosensor baseline step was applied before human mAbs were loaded onto anti-human IgG Fc capture sensor tips (AHC) by submerging sensor tips in 5 μg/mL human mAb until a response of 0.5 nm then washed in a kinetic buffer for 60 s. Association measurements were performed using a two-fold concentration gradient of parental PvRBP2b₁₆₉₋₄₇₀ and designs from 0.63-10 nM for 200 s and dissociation was measured in a kinetic buffer for 300 s. Sensor tips were regenerated using a cycle of 5 s in 100 mM glycine pH 1.5 and 5 s in kinetic buffer repeated five times. Baseline drift was corrected by subtracting the average shift of a human mAb loaded sensor not incubated with parental PvRBP2b₁₆₉₋₄₇₀ and designs, and an unloaded sensor incubated with parental PvRBP2b₁₆₉₋₄₇₀ and designs. Curve fitting analysis was performed with Octet Data Analysis 10.0 software using a global fit 1:1 model to determine K_{D} values and kinetic parameters. Curves that could not be reliably fitted were excluded from further analysis.

A multiplexed Luminex magnetic bead-based assay was utilized as previously described. Briefly, parental PvRBP2b₁₆₉₋₄₇₀, designs WHT2482, WHT2483 and WHT2484, and additional P. vivax antigens utilised in combination algorithms, were coupled to individual sets of internally labelled magnetic COOH beads, following bead activation using 50 mg/mL sulfo-NHS and 50 mg/mL EDC. Details on additional proteins and the amount coupled is provided in Table S3. Antigen-specific total IgG antibodies were then detected in plasma samples by incubating 50 μL coupled beads with 50 μL 1/100 dilution of human plasma, followed by addition of 1/100 detector antibody (PE conjugated anti-human IgG). On each plate, a 2-fold serial dilution from 1/50 to 1/25,600 of a positive control plasma pool (generated from adults from PNG) was included to enable conversion of the raw mean fluorescent intensity (MFI) to an arbitrary relative antibody unit (RAU) as previously described. Plates were read on a MAGPIX instrument as per the manufacturer's instruction, with data acquired from at least 15 beads per region.

The described multiplexed assay was used to measure antigen-specific IgG antibodies in plasma samples from malaria-endemic regions in Thailand and Brazil, as previously described. Briefly, yearlong cohort studies were conducted in Thailand (Kanchanaburi and Ratchaburi provinces) and Brazil (Manaus) across 2013-2014. Each site enrolled between 999 and 1,274 individuals with blood samples taken every month for qPCR detection of blood-stage P. vivax infections and plasma stored for antibody measurements. In the current study we measured total IgG antibody responses at the final visit of the yearlong cohort, enabling the magnitude to be related to the time since prior P. vivax infection.

Statistical approaches and classification algorithm are discussed below. The research included training and testing random forest classification algorithms using the antibody responses towards the panel of P. vivax antigens as predictor variables, and infection status as the outcome variable. Individuals infected within the previous nine months were classified as recently infected and those with a P. vivax infection prior to nine months, or no prior infection were classified as not recently infected. The research further included comparing the performance of a random forest classification algorithm trained on a panel of P. vivax antigens, whilst swapping the parental PvRBP2b₁₆₉₋₄₇₀ for designs WHT2482, WHT2483 and WHT2484 within this multi-antigen combination. The research further included assessing the classification performance using the area under the receiver operating curve (AUC).

The method, according to some embodiments of the present invention, allows the incorporation of information about the original polypeptide chain and/or the wild type (parental) protein. This information, which can be provided by various sources, in incorporated into the method as part of the rules by which amino acid substitutions are governed during the design procedure. Albeit optional, the addition of such information is advantageous as it reduces the probability of the method providing results which include folding-and/or function-abrogating substitutions.

To decrease the probability of sequences leading to misfolding during the sequence design process, residues that are known to be involved in structure stabilization, such as, residues that have an impact on correct folding (e.g., cysteines involved in disulfide bridges), necessary conformation change and allosteric communication with a functional site, and residues involved in posttranslational modifications, may be identified as “key residues”.

To further decrease the probability to reduce or abolish function during the sequence design process, residues that are known to be involved in any desired function or affect a desired attribute, may be identified as key residues. Positions occupied by key residues are regarded as unsubstitutable positions, and are fixed as the amino acid that occurs in the original polypeptide chain.

The term “key residues” refer to positions in the designed sequence that are defined in the rules as fixed (invariable), at least to some extent. Sequence positions which are occupied by key residues constitute a part of the unsubstitutable positions.

Information pertaining to key residues can be extracted, for example, from the structure of the original polypeptide chain (or the template structure), or from other highly similar structures when available. Exemplary criteria that can assist in identifying key residues, and support reasoning for fixing an amino-acid type or identity at any given position, include:

For enzymes catalyzing reactions of substrate molecules in an active site, key residues may be selected within a radius of about 5-8 Å around the substrate binding site, as may be inferred from complex crystal structures comprising a substrate, a substrate analog, an inhibitor and the like.

For metal binding proteins, key residues may be selected within about 5-8 Å around a metal atom.

Key residues may be selected within about 5-8 Å from any protein interface that involves the chain of interest in an oligomers, as interacting chains are oftentimes involved in dimerization interfaces, binding ligands or protein-substrates interactions.

Key residues may be selected within about 5-8 Å from DNA/RNA chains interacting with the protein of interest.

For proteins involved in immunogenicity, key residues may be selected within about 5-8 Å from the epitope region.

It is noted that the shape and size of the space within which key residues are selected is not limited to a sphere of a radius of 5-8 Å; the space can be of any size and shape that corresponds to the sequence, function and structure of the original protein.

It is further noted that specific key residues may be provided by any external source of information (e.g., a researcher).

The following section describes the data presented in figures illustrating aspects of the evaluation research (FIGS. 4; 5A-5C; 6A-6D; 7A-7C; 8A-8D; and 9-15).

FIG. 4 schematically illustrates design process of stabilized PvRBP2b₁₆₉₋₄₇₀ variants. Stabilized designs of PvRBP2b were computed under stringent conditions of minimally perturbing the solvent-accessible surfaces to maintain its antigenicity profile. Due to limited diversity of natural PvRBP2b homologs, in some designs we used AI-based ProteinMPNN (e.g., such as MPNN 120, described with reference to FIG. 2) to generate a pseudo-PSSM (e.g., PSSM 130A, as shown in FIG. 2) followed by PROSS atomistic design calculations (operations of sequence design module 140, structure modelling module 150, energy minimization module 160 and selection and output module 170, as described with reference to FIG. 2). A total of nine designs were tested.

FIGS. 5A-5C represent purification yields and biophysical characterization of parental PvRBP2b₁₆₉₋₄₇₀ and designs. FIG. 5A represents coomassie-stained SDS-PAGE gel of Ni-NTA affinity purified parental PvRBP2b₁₆₉₋₄₇₀ and nine designs in reducing conditions. FIG. 5B represents final yields (mg/L bacterial culture) for parental and stabilized designs after Ni-NTA affinity, ion exchange and size exclusion purification steps with fold change increase relative to parental yields shown on top of the corresponding bar graphs. The dotted line separates the two independent replicates for protein purification. FIG. 5C Dynamic light scattering (DLS) measurements of parental and three stabilized designs showing the hydrodynamic radius (Rₕ) and unfolding onset temperatures (Tₒₙₛₑₜ) from two independent replicates.

FIGS. 6A-6D represent structural comparison of parental PvRBP2b₁₆₉₋₄₇₀ with stabilized designs WHT2483 and 2484. FIG. 6A represents crystal structures of WHT2483 (purple) and WHT2484 (green) are overlayed with parental PvRBP2b₁₆₉₋₄₇₀ with RMSD values provided. FIG. 6B represents mutated residues are shown as spheres mapped onto ribbon representations of the parental PvRBP2b₁₆₉₋₄₇₀ structure. Mutations present in both WHT2483 and WHT2484 are shown in pink and mutations present only in WHT2484 are shown in green. Representative mutations that demonstrate stabilising effects are highlighted on the tertiary structure compared with parental PvRBP2b₁₆₉₋₄₇₀. FIG. 6C represents binding footprints of human antibodies against parental PvRBP2b₁₆₉₋₄₇₀. Parental PvRBP2b₁₆₉₋₄₇₀ is shown in surface representation (white) and coloured regions denote residues involved in antibody binding, with light chain interactions in a lighter shade and heavy chain interactions in a darker shade for each antibody. Interacting residues are obtained from previously published work and were determined using known method—PISA. Mutations within an antibody binding footprint are shown in blue for those in WHT2482-2484 inclusive (241242 Q378L) and teal for WHT2484 alone (237235 K248L and 283284 M324F). FIG. 6D represents total antibody bound surface (deep blue) for the eight human monoclonal antibodies is shown on parental PvRBP2b₁₆₉₋₄₇₀ surface (white).

FIGS. 7A-7C represent stabilized PvRBP2b₁₆₉₋₄₇₀ designs function as reliable serological markers for recent P. vivax infection. FIG. 7A represents antibody responses towards PvRBP2b proteins measured in relative antibody units (RAU) from the year-long cohort studies, as well as negative controls from Australian Red Cross (ARC), Brazil (Br Neg), Thai Red Cross (ThRC) and the Volunteer Blood Donor Registry (VBDR) in Victoria, Australia. Boxplots illustrate the median and 25^{th} and 75^{th} percentiles of the distribution of antibody responses for individuals who had a (i) current infection (i.e. positive qPCR results for P. vivax at the time of antibody measurement), (ii) recent infection within the previous nine months, (iii) old infection (i.e. infection nine to 12 months ago), (iv) no infection during the year-long cohort study, and (v) the negative controls. FIG. 7B represents correlation between parental PvRBP2b₁₆₉₋₄₇₀ and designs, and associated Pearson correlation coefficient (R), with colors representing the infection status as indicated. FIG. 7C represents receiver operating characteristic (ROC) curve for eight-antigen sero-diagnostic combination comparing parental PvRBP2b and designs, and the associated area under the curve (AUC).

FIGS. 8A-8D represent purification and label-free differential scanning fluorimetry of parental PvRBP2b₁₆₉₋₄₇₀ and three stabilized variants. FIG. 8A represents ion exchange chromatography (IEX) chromatograms and corresponding reduced SDS-PAGE gels of parental PvRBP2b₁₆₉₋₄₇₀ and designs. FIG. 8B represents size exclusion chromatography (SEC) chromatograms and corresponding reduced SDS-PAGE gels of parental PvRBP2b₁₆₉₋₄₇₀ and designs. FIG. 8C represents non-reduced (NR) and reduced (R) SDS-PAGE gel of recombinant parental PvRBP2b₁₆₉₋₄₇₀ and designs after affinity, IEX and SEC purification steps. FIG. 8D represents inflection temperatures (Tᵢ) of parental PvRBP2b₁₆₉₋₄₇₀ and designs using label free thermal shift analysis (Tycho NT.6, Nanotemper).

FIG. 9 represents electrostatics surfaces of PvRBP2b₁₆₉₋₄₇₀ and stabilized designs. Surface electrostatics calculations performed by the Adaptive Poisson-Boltzmann Solver (APBS) are displayed as blue (positive charge) and red (negative charge) formatting with a +/−5 KT/e range on 180° rotated surface representations of parental PvRBP2b₁₆₉₋₄₇₀, WHT2483, and WHT2484.

FIG. 10 represents representative BLI binding curves with PvRBP2b₁₆₉₋₄₇₀ designs and human monoclonal antibodies. Binding experiments were performed with five different concentrations from 0.6-10 nM of parental PvRBP2b₁₆₉₋₄₇₀ and stabilized designs. The measured binding curves are plotted (solid line) and fitted to a 1:1 binding model (dashed line). Representative binding curves are shown from two independent experiments. Corresponding K_{D} values are indicated.

Table 1 (show in FIG. 11) represents human antibody affinities to parental PvRBP2b₁₆₉₋₄₇₀ and three stabilized designs. Table 1 contains determined kinetic and affinity data from two independent experiments including values for K_{D}, kₐ, k_{d}, X², and R² values as measured by bio-layer interferometry. NB, non-binding.

Table S1 (shown in FIG. 12) represents mutated residues between parental PvRBP2b₁₆₉₋₄₇₀ and designs.

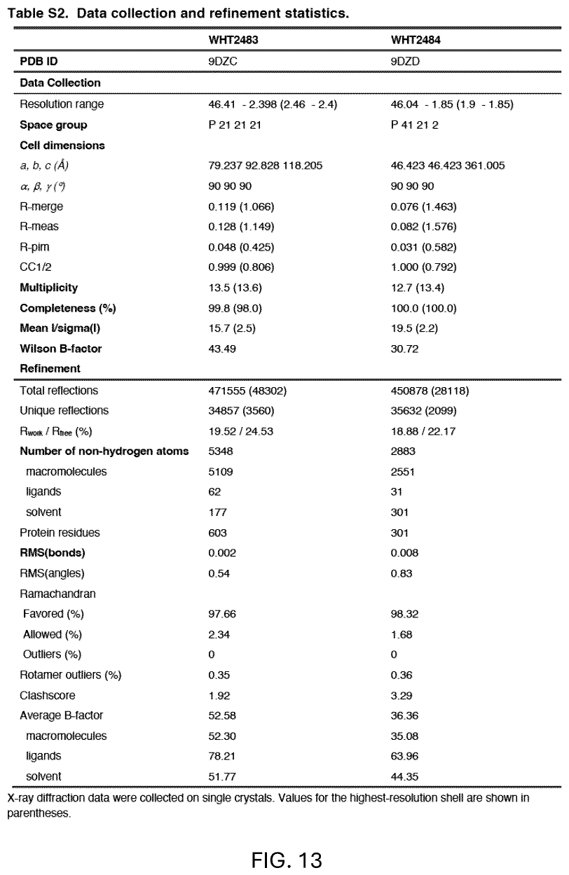

Table S2 (shown in FIG. 13) shows data collection and refinement statistics for WHT2483 and WHT2484.

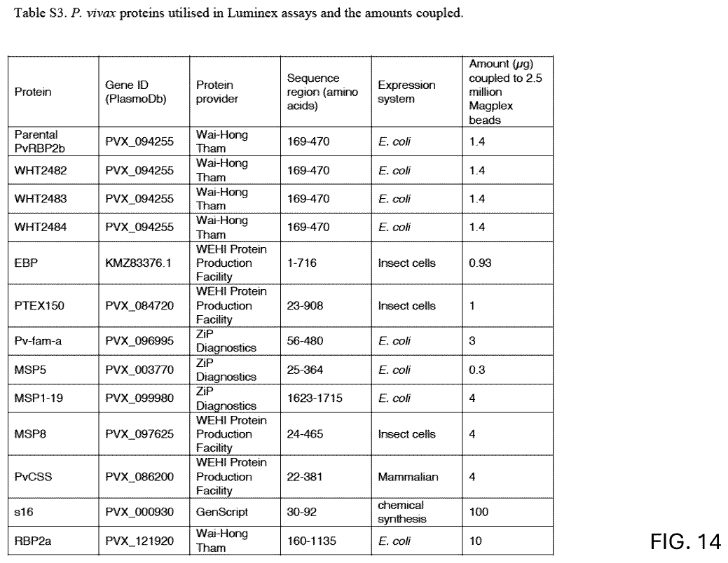

Table S3 (shown in FIG. 14) shows P. vivax proteins utilized in Luminex assays and the amounts coupled.

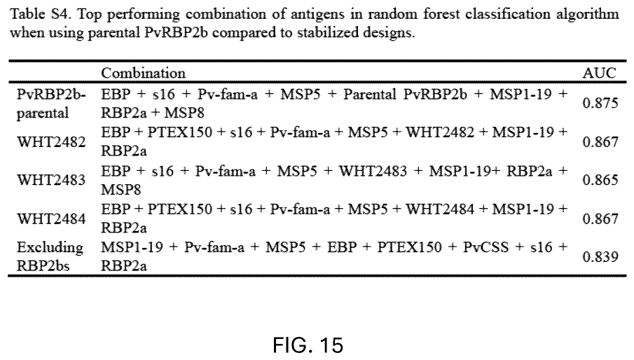

Table S4 (shown in FIG. 15) shows top performing combination of antigens in random forest classification algorithm when using parental PvRBP2b compared to stabilized designs.

As can be seen in the provided description, the present invention represents a system and method for producing a modified protein that provides an improvement of the technological field of protein design and stabilization by providing tools that enable the efficient design and production of proteins with enhanced stability characteristics compared to the parental protein, while retaining immunogenicity to naturally acquired human monoclonal antibodies. In particular, the suggested invention is efficiently applicable to proteins with large and topologically complex structures that have limited natural diversity of homologs and circumvent the requirement for a deep multiple-sequence alignment. The suggested invention may potentially help overcoming barriers to the implementation of economical and resilient diagnostics and vaccine immunogens for many infectious diseases. Unless explicitly stated, the method embodiments described herein are not constrained to a particular order or sequence. Furthermore, all formulas described herein are intended as examples only and other or different formulas may be used. Additionally, some of the described method embodiments or elements thereof may occur or be performed at the same point in time.

While certain features of the invention have been illustrated and described herein, many modifications, substitutions, changes, and equivalents may occur to those skilled in the art. It is, therefore, to be understood that the appended claims are intended to cover all such modifications and changes as fall within the true spirit of the invention.

Various embodiments have been presented. Each of these embodiments may of course include features from other embodiments presented, and embodiments not specifically described may include various features described herein.

1. A method of producing a modified protein comprising at least one modified polypeptide chain having amino acid substitutions relative to an original polypeptide chain in a parental protein, the method comprising:
generating a protein expression vector suitable for use in a protein expression system by:
(i) determining a plurality of backbone features characterizing a backbone of the parental protein or a segment thereof;
(ii) inferring a pretrained encoder-decoder-based message passing neural network (MPNN) on the determined plurality of backbone features to predict a probability for an amino acid to be located at a specific position of the backbone;
(iii) based on the predicted probability, defining a position-specific scoring matrix (PSSM) characterizing, per each position in the original polypeptide chain, amino acid alternatives;
(iv) combinatorically generating a plurality of designed sequences, each of said designed sequences corresponds to a modified polypeptide chain and comprises one or more amino acid substitutions each being one of said amino acid alternatives, and threading each of said designed sequences on a template structure of said original polypeptide chain, to thereby generate a plurality of designed structures;
(v) sorting said plurality of designed structures according to a minimized energy scoring, said minimized energy scoring is determined by subjecting each of said designed structures to an energy minimization; and
(vi) selecting at least one of said plurality of designed structures, corresponding to said modified polypeptide chain, based on said minimized energy scoring, thereby obtaining an amino acid sequence of said modified polypeptide chain for use as sequence data input for providing said protein expression vector; and
expressing the modified protein in said protein expression system using said protein expression vector, thereby producing the modified protein.

2. The method of claim 1, wherein said substitutions improve the stability of the modified protein relative to the parental protein, as determined by at least one of:
a thermal denaturation temperature of the modified protein being equal or higher than a thermal denaturation temperature of the parental protein;
a solubility of the modified protein being equal or higher than a solubility of the parental protein;
a degree of misfolding of the modified protein being equal or lower than a degree of misfolding of the parental protein;
a half-life of the modified protein being equal or longer than a half-life of the parental protein;
a specific activity of the modified protein being equal or higher than a specific activity of the parental protein; and
a recombinant expression level of the modified protein being equal or higher than a recombinant expression level of the parental protein.

3. The method of claim 1, wherein said determining a plurality of backbone features characterizing a backbone of the parental protein comprises:
determining the coordinates of one or more backbone atoms of a protein, wherein the backbone atoms include at least one of: a nitrogen (N) atom, a carbon (C) atom, an oxygen (O) atom, an alpha carbon (Cα) atom, and a virtual beta carbon (Cβ);
based on the determined coordinates, constructing a graph representation of the backbone, said graph representation having nodes representing amino acids of the parental protein, and edges defined by the backbone features, said backbone features being formed based on at least one of: interatomic distances related to adjacent nodes; characteristics of relative atomic frame orientations and rotations between adjacent nodes; backbone dihedral angles in the adjacent nodes.

4. The method of claim 3, wherein said inferring the MPNN comprises:
preprocessing the constructed graph representation to make the nodes thereof dissociated from the amino acids of the parental protein;
using a message-passing algorithm to encode the pre-processed graph representation, thereby obtaining an encoded graph representation comprising latent information about a structure of the backbone, said message-passing algorithm having messages constructed by applying a pretrained multilayer perception (MLP) model on each pair of the adjacent nodes and edges therebetween;
applying a decoding algorithm on the encoded graph representation, to predict, per each node and each amino acid, a probability of a respective node to be associated with a respective amino acid, thereby predicting the probability for the amino acid to be located at the specific position of the backbone.

5. The method of claim 1, wherein said defining the PSSM comprises:
logarithmically normalizing the predicted probabilities;
averaging the normalized probabilities over a plurality of independent initializations, to account for stochastic variations; and
defining the PSSM based on the averaged normalized probabilities.

6. The method of claim 1, wherein a selected modified polypeptide chain corresponds to designed structure having a minimal value for said minimized energy scoring.

7. The method of claim 6, wherein said energy minimization is a global energy minimization.

8. The method of claim 1, wherein said plurality of designed sequences is combinatorically generated under an acceptance threshold based on said stability scoring.

9. The method of claim 1, wherein said at least one modified polypeptide chain comprises at least six amino acid substitutions relative to said original polypeptide chain.

10. The method of claim 1, wherein said energy minimization comprises at least one operation selected from the group consisting of bond length optimization, bond angle optimization, backbone dihedral angles optimization, amino acid side-chain packing optimization, rigid-body optimization, and sidechain dihedral angle minimization of the modified polypeptide chain.

11. A system for producing a modified protein comprising at least one modified polypeptide chain having amino acid substitutions relative to an original polypeptide chain in a parental protein, the system comprising: at least one non-transitory memory device, wherein modules of instruction code are stored, and at least one processor associated with said at least one memory device, and configured to execute the modules of instruction code, whereupon execution of said modules of instruction code, the at least one processor is configured to:
generate a protein expression vector suitable for use in a protein expression system by:
(i) determining a plurality of backbone features characterizing a backbone of the parental protein or a segment thereof;
(ii) inferring a pretrained encoder-decoder-based message passing neural network (MPNN) on the determined plurality of backbone features to predict a probability for an amino acid to be located at a specific position of the backbone;
(iii) based on the predicted probability, defining a position-specific scoring matrix (PSSM) characterizing, per each position in the original polypeptide chain, amino acid alternatives;
(iv) combinatorically generating a plurality of designed sequences, each of said designed sequences corresponds to a modified polypeptide chain and comprises one or more amino acid substitutions each being one of said position-specific amino acid alternatives, and threading each of said designed sequences on a template structure of said original polypeptide chain, to thereby generate a plurality of designed structures;
(v) sorting said plurality of designed structures according to a minimized energy scoring, said minimized energy scoring is determined by subjecting each of said designed structures to an energy minimization; and
(vi) selecting at least one of said plurality of designed structures, corresponding to said modified polypeptide chain, based on said minimized energy scoring, thereby obtaining an amino acid sequence of said modified polypeptide chain for use as sequence data input for providing said protein expression vector; and
express the modified protein in said protein expression system using said protein expression vector, thereby producing the modified protein.

12. The system of claim 11, wherein said substitutions improve the stability of the modified protein relative to the parental protein, as determined by at least one of:
a thermal denaturation temperature of the modified protein being equal or higher than a thermal denaturation temperature of the parental protein;
a solubility of the modified protein being equal or higher than a solubility of the parental protein;
a degree of misfolding of the modified protein being equal or lower than a degree of misfolding of the parental protein;
a half-life of the modified protein being equal or longer than a half-life of the parental protein;
a specific activity of the modified protein being equal or higher than a specific activity of the parental protein; and
a recombinant expression level of the modified protein being equal or higher than a recombinant expression level of the parental protein.

13. The system of claim 11, wherein said at least one processor is configured to determine a plurality of backbone features characterizing a backbone of the parental protein by:
determining the coordinates of one or more backbone atoms of a protein, wherein the backbone atoms include at least one of: a nitrogen (N) atom, a carbon (C) atom, an oxygen (O) atom, an alpha carbon (Cα) atom, and a virtual beta carbon (Cβ);
based on the determined coordinates, constructing a graph representation of the backbone, said graph representation having nodes representing amino acids of the parental protein, and edges defined by the backbone features, said backbone features being formed based on at least one of: interatomic distances related to adjacent nodes; characteristics of relative atomic frame orientations and rotations between adjacent nodes; backbone dihedral angles in the adjacent nodes.

14. The system of claim 13, wherein said at least one processor is configured to infer the MPNN by:
preprocessing the constructed graph representation to make the nodes thereof dissociated from the amino acids of the parental protein;
using a message-passing algorithm to encode the pre-processed graph representation, thereby obtaining an encoded graph representation comprising latent information about a structure of the backbone, said message-passing algorithm having messages constructed by applying a pretrained multilayer perception (MLP) model on each pair of the adjacent nodes and edges therebetween; and
applying a decoding algorithm on the encoded graph representation, to predict, per each node and each amino acid, a probability of a respective node to be associated with a respective amino acid, thereby predicting the probability for the amino acid to be located at the specific position of the backbone.

15. The system of claim 11, wherein said at least one processor is configured to define the PSSM by:
logarithmically normalizing the predicted probabilities;
averaging the normalized probabilities over a plurality of independent initializations, to account for stochastic variations; and
defining the PSSM based on the averaged normalized probabilities.

16. The system of claim 11, wherein a selected modified polypeptide chain corresponds to designed structure having a minimal value for said minimized energy scoring.

17. The system of claim 16, wherein said energy minimization is a global energy minimization.

18. The system of claim 11, wherein said plurality of designed sequences is combinatorically generated under an acceptance threshold based on said stability scoring.

19. The system of claim 11, wherein said at least one modified polypeptide chain comprises at least six amino acid substitutions relative to said original polypeptide chain.

20. The system of claim 11, wherein said at least one processor is further configured to perform the energy minimization by at least one operation selected from the group consisting: of bond length optimization, bond angle optimization, backbone dihedral angles optimization, amino acid side-chain packing optimization, rigid-body optimization, and sidechain dihedral angle minimization of the modified polypeptide chain.
